(19) United States
(12) Patent Application Publication
(10) Pub. No.: US 2008/0262610 A1
(43) Pub. Date: Oct. 23, 2008

(54) BIOMECHANICAL DESIGN OF INTRACORNEAL INLAYS

(76) Inventors: Alan Lang, Long Beach, CA (US); Troy Miller, Rancho Santa Margarita, CA (US); Ned Schneider, Aliso Viejo, CA (US); Alexander Vatz, Lake Forest, CA (US); Tonya Brooke Icenogle, Newport Beach, CA (US); Sylvia Franz, Laguna Beach, CA (US); Derrick Johnson, Orange, CA (US)

Correspondence Address: SHAY GLENN LLP, 2755 CAMPUS DRIVE, SUITE 210, SAN MATEO, CA, 94403, US

(21) Appl. No.: 11/738,349

(22) Filed: Apr. 20, 2007

(51) Int. Cl. A61F 2/14

(52) U.S. Cl. 623/5.16

(57) ABSTRACT

Provided herein are intracorneal inlays for correcting vision impairments by altering the shape of the anterior corneal surface. The physical design of the inlay to induce the desired change of the anterior corneal surface includes consideration of the biomechanical response of the corneal tissue to the physical shape of the inlay. This biomechanical response can differ depending on the thickness, diameter, and profile of the inlay. In one embodiment, inlays having diameters smaller than the pupil are provided for correcting presbyopia. To provide near vision, an inlay is implanted centrally in the cornea to induce an “effect” zone on the anterior corneal surface, within which diopter power is increased. Distance vision is provided by a region of the cornea peripheral to the “effect” zone. In another embodiment, small diameter inlays are provided that induce “effect” zones on the anterior corneal surface that are much larger in diameter than the inlays.

### FIELD OF THE INVENTION

The field of the invention relates generally to corneal implants, and more particularly, to intracorneal inlays.

### BACKGROUND INFORMATION

As is well known, abnormalities in the human eye can lead to vision impairment. Some typical abnormalities include variations in the shape of the eye, which can lead to myopia (near-sightedness), hyperopia (farsightedness) and astigmatism as well as variations in the tissue present throughout the eye, such as a reduction in the elasticity of the lens, which can lead to presbyopia. A variety of technologies have been developed to try and address these abnormalities, including corneal implants.

Corneal implants can correct vision impairment by altering the shape of the cornea. Corneal implants can be classified as an onlay or an inlay. An onlay is an implant that is placed over the cornea such that the outer layer of the cornea, e.g., the epithelium, can grow over and encompass the implant. An inlay is an implant that is surgically implanted into the cornea beneath a portion of the corneal tissue by, for example, cutting a flap in the cornea and inserting or placing the inlay beneath the flap. Both inlays and outlays can alter the refractive power of the cornea by changing the shape of the anterior cornea, by having a different index of refraction than the cornea, or both. Since the cornea is the strongest refracting optical element in the human ocular system, altering the cornea's anterior surface is a particularly useful method for correcting vision impairments caused by refractive errors.

Inlays are also useful for correcting other visual impairments including presbyopia. Three common approaches are used with other ophthalmic devices (e.g., contact lenses, intraocular lenses, LASIK, etc.). In a monovision prescription, the diopter power of one eye is adjusted to focus distance objects and the power of the other eye is adjusted to focus near objects. Thus, the appropriate eye is used to clearly view the object of interest. In the next two approaches, multifocal or bifocal optics are used to simultaneously (in one eye) provide powers to focus both distant and near objects. One common multifocal design includes a central zone of higher diopter power to focus near objects, surrounded by a peripheral zone of the desired lower power to focus distance objects. In a modified monovision prescription, the diopter power of one eye is adjusted to focus distance objects, and in the fellow eye a multifocal optical design is induced by the intracorneal inlay. Thus, the subject has the necessary diopter power from both eyes to view distant objects. The near power zone of the multifocal eye provides the necessary power for viewing near objects. In a bilateral multifocal prescription, the multifocal optical design is induced in both eyes by intracorneal inlays. Both eyes contribute to both distance and near vision.

Whether the intracorneal inlay design induces a multi-diopter power multifocal effect for the correction of presbyopia or a single diopter power for the correction of simple refractive error, it is necessary to understand the biomechanical response of the cornea to the presence of the inlay to properly design the physical shape of the inlay to induce the desired optical effect elicited by a change in the anterior surface of the cornea. Watsky, et al, proposed a simple biomechanical response in Investigative Ophthalmology and Visual Science, vol. 26, pp. 240-243 (1985). In this biomechanical model (“Watsky model”), the anterior corneal surface radius of curvature is assumed to be equal to the thickness of the lamellar corneal material (i.e., flap) between the anterior corneal surface and the anterior surface of the intracorneal inlay plus the radius of curvature of the anterior surface of the inlay. Lang, et al, demonstrated a more accurate biomechanical model based on the assumption that the thickness profile of the intracorneal inlay is transferred to the anterior corneal surface through an analysis of clinical refractive data with 5 mm diameter intracorneal inlays; “First order design of intracorneal inlays: dependence on keratometric flap and corneal properties,” ARVO abstracts 2006, poster #3591. The design method based on this biomechanical model is described in U.S. patent application Ser. No. 11/293644, filed on Dec. 1, 2005, the specification of which is herein incorporated by reference. Prior publications and patents disclose designs of relatively large diameter inlays for the correction of either hyperopic or myopic refractive error. See U.S. Pat. No. 5,123,921 by Werblin and the references cited therein. Reviews of clinical outcomes for implanted inlays or methods for design generally discuss relatively thick inlays (e.g., greater than 200 microns) for which the above simple biomechanical response models have some validity. This is because the physical size of the inlay dominates the biomechanical response and dictates the primary anterior surface change. However, when the inlay is relatively small and thin, the material properties of the lamellar flap and corneal bed contribute significantly to the resulting change in the anterior corneal surface. Therefore methods of inlay design should account for this biomechanical response to be effective in causing the desired anterior surface change, whether producing a monofocal or multifocal effect.

### SUMMARY

Provided herein are intracorneal inlays for correcting vision impairments by altering the shape of the anterior corneal surface. The physical design of the intracorneal inlay to induce the desired change of the anterior corneal surface includes consideration of the biomechanical response of the corneal tissue to the physical shape of the inlay. This biomechanical response can differ depending on the thickness, diameter, and profile of the inlay.

In one embodiment, inlays having diameters smaller than the diameter of the pupil are provided for correcting presbyopia. To provide near vision, an inlay is implanted centrally in the cornea to induce an “effect” zone on the anterior corneal surface that is smaller than the optical zone of the cornea, wherein the “effect” zone is the area of the anterior corneal surface affected by the inlay. The implanted inlay increases the curvature of the anterior corneal surface within the “effect” zone, thereby increasing the diopter power of the cornea within the “effect” zone. Because the inlay's “effect” zone is also smaller than the diameter of the pupil, light rays from distance objects by-pass the inlay and refract using the region of the cornea peripheral to the “effect” zone to create an image of the distant objects on the retina.

The small diameter inlays may be used alone or in conjunction with other refractive procedures. In an embodiment, a small diameter inlay is used in conjunction with LASIK for correcting myopia or hyperopia. In this embodiment, a LASIK procedure is used to correct for distance refractive error and the small diameter inlay is used to provide near vision for presbyopic subjects. This embodiment is applied either to one eye as in modified monovision or applied to both eyes.

In another embodiment, a larger diameter and thicker inlay is used to correct hyperopia or other refractive error. The “effect” zone of the larger inlay extends beyond the largest pupil diameter typically experienced by the implanted subject. Thus, the entire optical zone of the cornea is altered to correct the refractive errors.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention not be limited to the details of the example embodiments.

### BRIEF DESCRIPTION OF THE FIGURES

FIG. 1 is a cross-sectional view of a cornea showing an intracorneal inlay implanted in the cornea according to an embodiment of the invention.

FIG. 2 is a cross-sectional view of a cornea showing an intracorneal inlay with a finite edge thickness implanted in the cornea according to an embodiment of the invention.

FIG. 3 is a diagram of an eye illustrating the use of a small diameter inlay to provide near vision according to an embodiment of the invention.

FIGS. 4-6 show the anterior corneal height change in human subjects implanted with three different inlay designs.

FIG. 7 plots the diameters of the corneal “effect” zones for human subjects implanted with 1.5 mm and 2.0 mm diameter inlays.

FIG. 8 plots the diameters of the corneal “effect” zones for human subjects implanted with inlays of different edge thicknesses.

FIG. 9 plots the diameters of the corneal “effect” zones for human subjects implanted with inlays of different center thicknesses.

FIG. 10 plots estimates of the spherical refractive power change for human subjects implanted with inlays as a function of the theoretically expected refractive change.

FIG. 11 plots near visual acuity for human subjects implanted with inlays of different center thicknesses.

FIG. 12 plots distance visual acuity for human subjects implanted with inlays of different center thicknesses.

FIG. 13 is a cross-sectional view of an inlay with a taper region according to an embodiment of the invention.

FIG. 14 is a cross-sectional view of an inlay with a taper region according to another embodiment of the invention.

FIG. 15 is a cross-sectional view of an inlay with a taper region according to another embodiment of the invention.

FIG. 16 is a cross-sectional view of an inlay with a taper region according to another embodiment of the invention.

FIG. 17 is a cross-sectional view of an inlay with a taper region according to another embodiment of the invention.

FIG. 18 is a cross-sectional view of an inlay with a taper region according to another embodiment of the invention.

FIG. 19 is a cross-sectional view of an inlay with a taper region according to another embodiment of the invention.

FIG. 20 is a cross-sectional view of a cornea showing a larger diameter intracorneal inlay with a finite edge thickness implanted in the cornea according to another embodiment of the invention.

FIG. 21 shows a correlation between measured change in corneal power and expected change in corneal power for large diameter inlays according to an embodiment of the invention.

FIG. 22 shows a cross-sectional view of a thickness profile defined by a difference between a desired anterior corneal surface and the pre-implant anterior corneal surface.

FIG. 23 shows the chemical structures for the monomer precursors of an inlay material according to embodiment of the invention.

FIG. 24 shows an apparatus for forming the inlay material according to an embodiment of the invention.

FIG. 25 is a temperature chart showing temperature over time for a polymerization process according to an embodiment of the invention.

### DETAILED DESCRIPTION

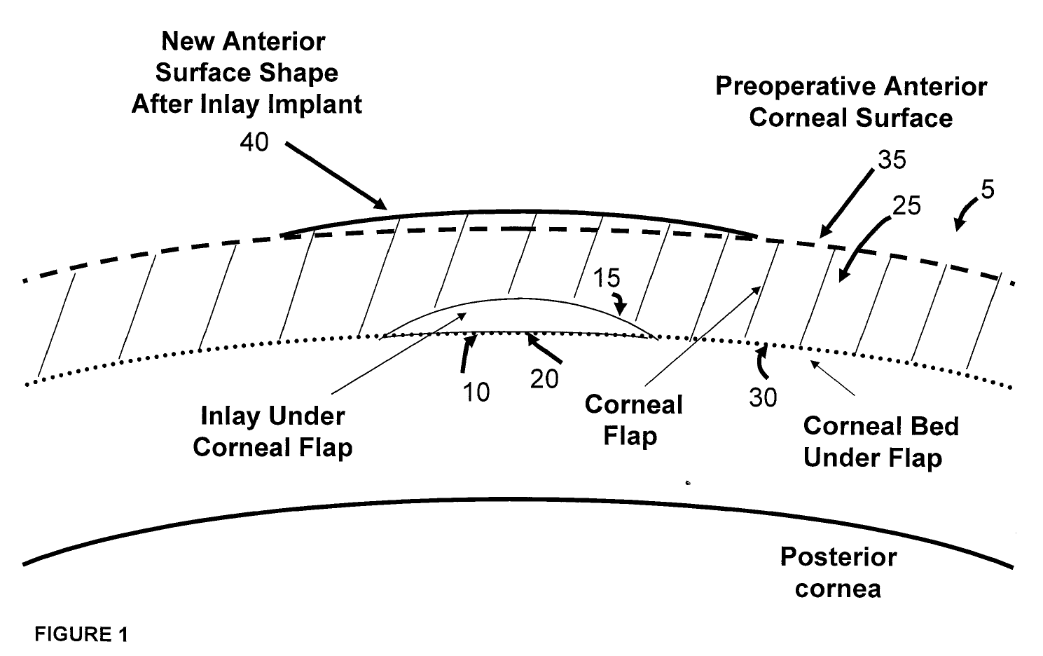

FIG. 1 show an example of an intracorneal inlay 10 implanted in a cornea 5. The inlay 10 may have a meniscus shape with an anterior surface 15 and a posterior surface 20. The inlay 10 is preferably implanted in the cornea at a depth of 50% or less of the cornea thickness (approximately 250 μm or less), and is placed on the stromal bed 30 of the cornea created by a keratome. The inlay 10 may be implanted in the cornea 5 by cutting a flap 25 into the cornea, lifting the flap 25 to expose the cornea's interior, placing the inlay 10 on the exposed area of the cornea's interior with the inlay centered on the subject's pupil or visual axis, and repositioning the flap 25 over the inlay 10. The flap 25 may be cut using a laser, e.g., a femtosecond laser, a mechanical keratome or manually by an ophthalmic surgeon. When the flap 25 is cut into the cornea, a small section of corneal tissue is left intact to create a hinge for the flap 25 so that the flap 25 can be repositioned accurately over the inlay 20. After the flap 25 is repositioned over the inlay, the cornea heals around the flap 25 and seals the flap 25 back to the un-cut peripheral portion of the anterior corneal surface. Alternatively, a pocket or well having side walls or barrier structures may be cut into the cornea, and the inlay inserted between the side walls or barrier structures through a small opening or “port” in the cornea. The inlay should be positioned on the corneal bed with the inlay centered on the subject's pupil or visual axis. Several methods for forming flaps in corneal tissue, and other related information, are described in further detail in co-pending U.S. patent application Ser. No. 10/924,152, filed Aug. 23, 2004, entitled “Method for Keratophakia Surgery,” which is fully incorporated by reference herein.

The inlay is delivered to the stromal bed 30 by means of an injector, inserter or spoon on which the inlay may be stored. The insertion system delivers the inlay in a hydrated state, and maintains the proper inlay orientation such that the inlay's posterior surface rests on the cornea's convex bed. Delivery systems are required separately for an open flap or pocket. Additionally the inlay delivery system should be easy for the surgeon to use and introduce a minimum amount of saline or visco elastic onto the corneal bed during the procedure.

The inlay 10 changes the refractive power of the cornea by altering the shape of the anterior corneal surface. In FIG. 1, the pre-operative anterior corneal surface is represented by dashed line 35 and the post-operative anterior corneal surface induced by the underlying inlay 10 is represented by solid line 40.

The inlay may have properties similar to those of the cornea (e.g., index of refraction around 1.376, water content of 78%, etc.), and may be made of hydrogel or other clear biocompatible material. Materials that can be used for the inlay include, but are not limited to, Lidofilcon A, Poly-HEMA (hydroxyethyl methylacrylate), poly sulfone, silicone hydrogel, and the like. Such materials cover compositions ranging from 20% to 50% HEMA (hydroxyethyl methylacrylate), 85% to 30% NVP (normal vinyl pyrrolidone), and/or 0% to 25% PVP (polyvinyll pyrrolidone). Other formulations of such materials cover compositions ranging from 15% to 50% MMA (methyl methylacrylate), 85% to 30% NVP (normal vinyl pyrrolidone), and/or 0% to 25% PVP (polyvinyll pyrrolidone). The water content of these compositions can range from 65% to 80%. In the preferred embodiment, the material is composed of 78% NVP and 22% MMA(methyl methacrylate), with allymethacrylate as the crosslinker and AIBN (azobisisobutylonitrile) as the initiator. Additional details on the inlay material is provided in Appendix A. In the preferred embodiment, the inlay has an index of refraction of approximately 1.376±.0.008, which is substantially the same as the cornea. As a result, the inlay has no intrinsic diopter power.

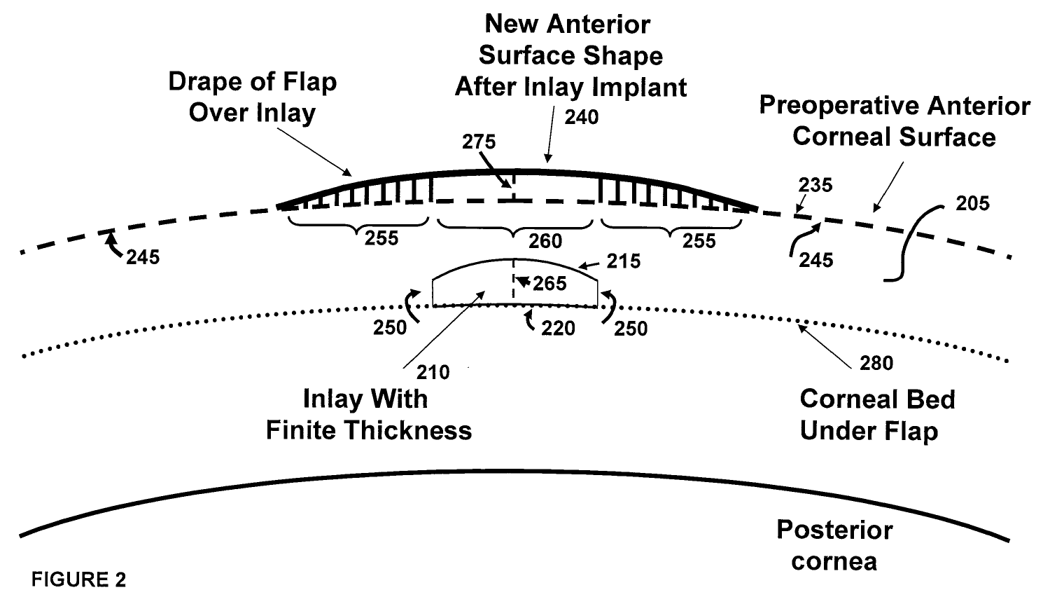

The biomechanical effect governing the shape of the post-operative anterior corneal 240 surface in response to an inlay 210 with finite center thickness 265 and edge thickness 250 is shown in FIG. 2, in which the “effect” zone extends beyond the diameter of the inlay 210. The “effect” zone is composed of the geometric projection of the inlay diameter on the anterior surface 260 and a “drape” of the flap 255 peripheral to the projection of the inlay diameter. The diameter of the “effect” zone is a function of the inlay center thickness 265, the inlay diameter, the inlay edge thickness 250, and the radius of curvature of the anterior surface 215. The center thickness of the “effect” zone 275 is less than the center thickness of the inlay 265. This is because the viscoelastic properties of the corneal flap material allow absorption of some of the inlay volume, resulting in less of the inlay volume “pushing through” the flap to the anterior surface. The viscoelastic properties of the corneal flap may also cause some localized flow of the displace volume, adding to the drape region 255 of the anterior surface change. The degree to which the anterior surface shape 240 differs from the inlay shape 215 is a complicated function of the inlay diameter, center thickness 265, anterior surface 215 radius of curvature, edge thickness 250 and the biomechanical properties of the lamellar flap material. This relationship can be found by sophisticated modeling such a Finite Element Analysis (FEA) or empirically by in vitro or in vivo experiments. With the latter, the changes in animal model corneal surfaces or human corneal surfaces in a clinical trial are correlated with the inlay design implanted.

The desired change in diopter power and image quality of the inlay effect is a function of the shape and curvature of the post-operative anterior corneal surface 240. Because the anterior corneal surface 240 is not necessarily a simple geometric projection of the inlay shape, as has been discussed in prior publications and patents, knowledge of this relationship is important to the effective design of the intracorneal inlay.

### Small Diameter Intracorneal Inlays for the Correction of Presbyopia

This section discusses the use of small intracorneal inlays having diameters that are small in comparison with the pupil for correcting presbyopia. In the preferred embodiment, a small inlay (e.g., 1 to 2 mm in diameter) is implanted centrally in the cornea to induce an “effect” zone (e.g., 2 to 4 mm in diameter) on the anterior corneal surface that is smaller than the optical zone of the cornea for providing near vision. Here, “effect” zone is the area of the anterior corneal surface altered by the inlay. The implanted inlay increases the curvature of the anterior corneal surface within the “effect” zone, thereby increasing the diopter power of the cornea within the “effect” zone. Distance vision is provided by the region of the cornea peripheral to the “effect” zone.

Presbyopia is characterized by a decrease in the ability of the eye to increase its power to focus on nearby objects due to a loss of elasticity in the crystalline lens with age. Typically, a person suffering from Presbyopia requires reading glasses to provide near vision. For early presbyopes (e.g., about 45 to 55 years of age), at least 1 diopter is typically required for near vision. For complete presbyopes (e.g., about 60 years of age or older), between 2 and 3 diopters of additional power is required.

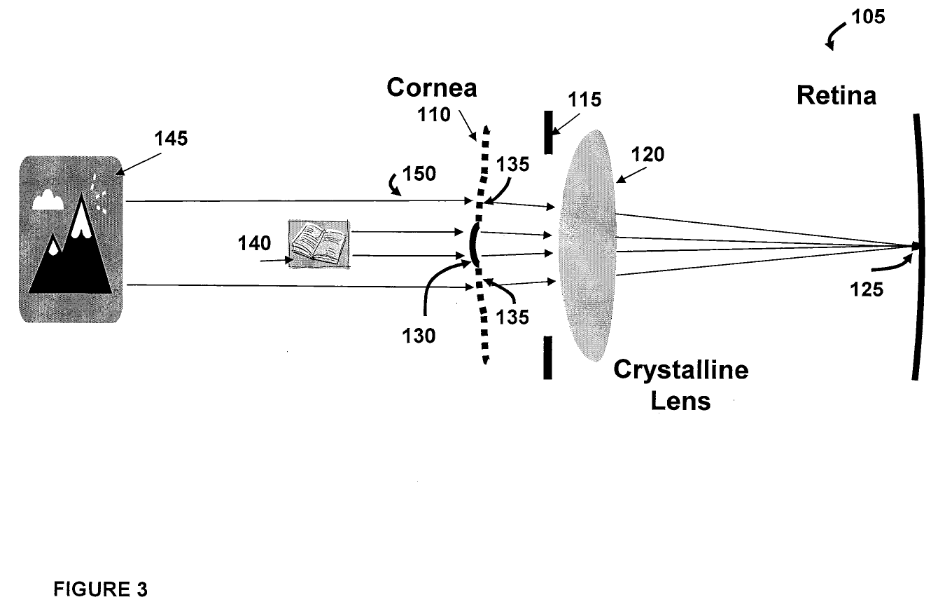

FIG. 3 shows an example of how a small inlay can provide near vision to a subject's eye while retaining some distance vision according to an embodiment of the invention. The eye 105 comprises the cornea 110, the pupil 115, the crystalline lens 120 and the retina 125. In this example, the small inlay (not shown) is implanted centrally in the cornea to create a small diameter “effect” zone 130. Both the inlay diameter and effect zone diameter 130 are smaller than the pupil diameter 115. The “effect” zone 130 provides near vision by increasing the curvature of the anterior corneal surface, and therefore the diopter power within the “effect” zone 130. The region 135 of the cornea peripheral to the “effect” zone provides distance vision.

An advantage of the small intracorneal inlay is that when concentrating on nearby objects 140, the pupil naturally becomes smaller (e.g., near point miosis) making the inlay effect even more effective. Further increases in the inlay effect can be achieved by simply increasing the illumination of a nearby object (e.g., turning up a reading light).

Because the “effect” zone 130 is smaller than the diameter of the pupil 115, light rays 150 from distant objects 145 by-pass the inlay and refract using the region of the cornea peripheral to the “effect” zone to create an image of the distant objects on the retina 125, as shown in FIG. 3. This is particularly true with larger pupils. At night, when distance vision is most important, the pupil naturally becomes larger, thereby reducing the inlay effect and maximizing distance vision.

A subject's natural distance vision is in focus only if the subject is emmetropic (i.e., does not require glasses for distance vision). Many subjects are ammetropic, requiring either myopic or hyperopic refractive correction. Especially for myopes, distance vision correction can be provided by myopic Laser in Situ Keratomileusis (LASIK) or other similar corneal refractive procedures. After the distance corrective procedure is completed, the small inlay can be implanted in the cornea to provide near vision. Since LASIK requires the creation of a flap, the inlay may be inserted concurrently with the LASIK procedure. The inlay may also be inserted into the cornea after the LASIK procedure since the flap can be re-opened. Therefore, the small inlay may be used in conjunction with other refractive procedures, such as LASIK for correcting myopia or hyperopia.

A method for designing a small inlay to provide near vision will now be described. FIG. 2 shows a small inlay 210 implanted in the cornea 205 and the change in the shape of the anterior corneal surface 240 induced by the inlay 210. In FIG. 2, the pre-implant anterior corneal surface is represented by dashed line 235 and the post-implant anterior corneal surface induced by the inlay 210 is represented by solid line 240. The inlay 210 does not substantially affect the shape of the anterior corneal surface in the region of the cornea 245 peripheral to the “effect” zone so that distance vision is undisturbed in the peripheral 245. In the case where a distance corrective procedure is performed prior to implantation of the inlay, the pre-implant anterior corneal surface 235 is the anterior corneal surface after the distance corrective procedure but before implantation of the inlay.

The inlay 210 has a finite edge thickness 250. The edge thickness 250 can not be made zero due to the finite material properties of the inlay. The finite edge thickness 250 of the inlay may contribute to the draping effect, as described below. To minimize the draping effect, the edge thickness 250 of the inlay 210 can be made as small as possible, e.g., less than about 20 microns. In addition to a finite edge thickness 250, the inlay may have a tapered region (not shown) that tapers downward from the anterior surface 215 of the inlay to the edge 250 of the inlay. The tapered region is discussed in more detail below.

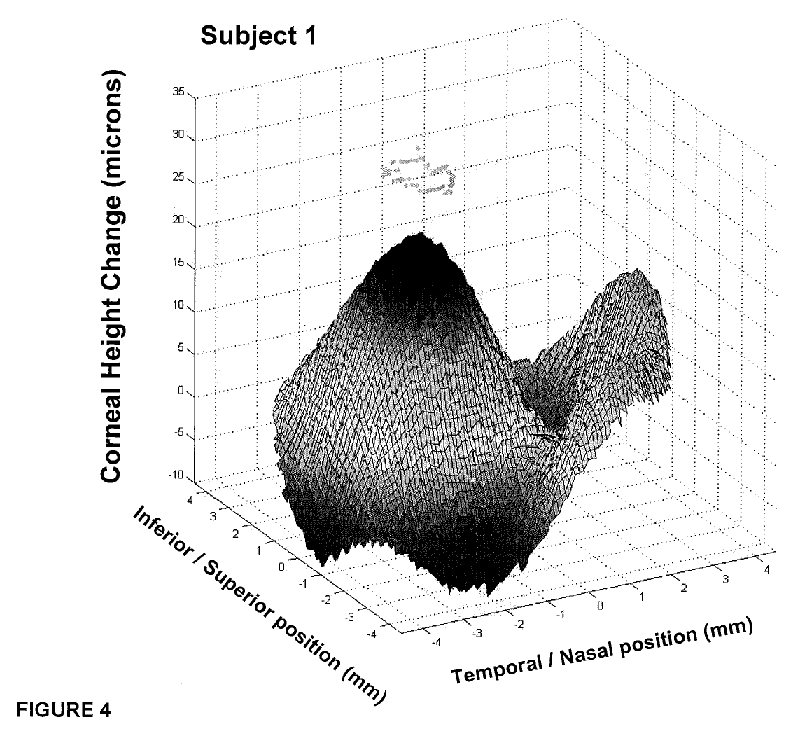

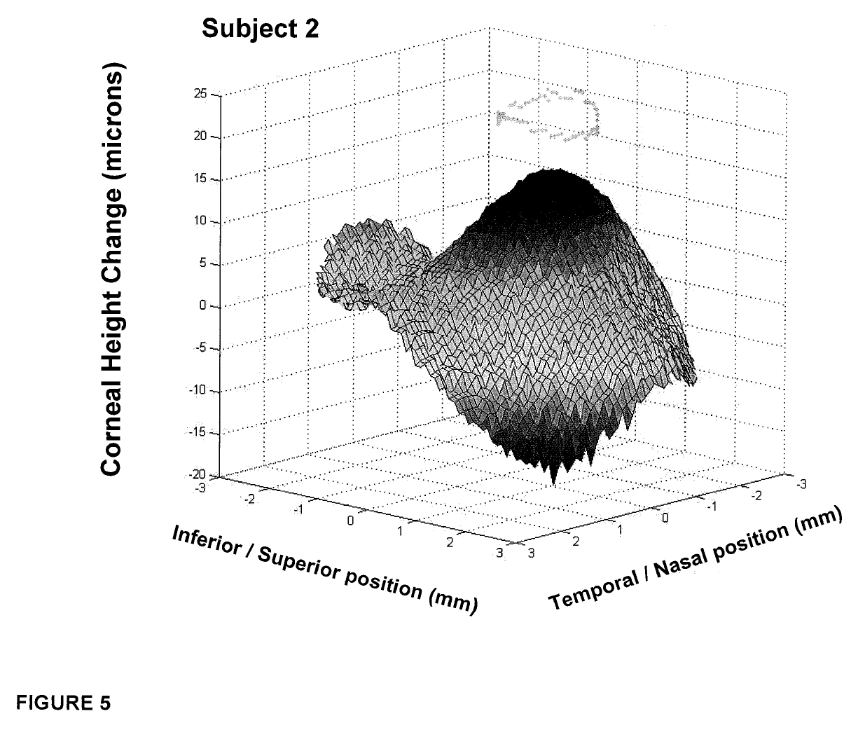

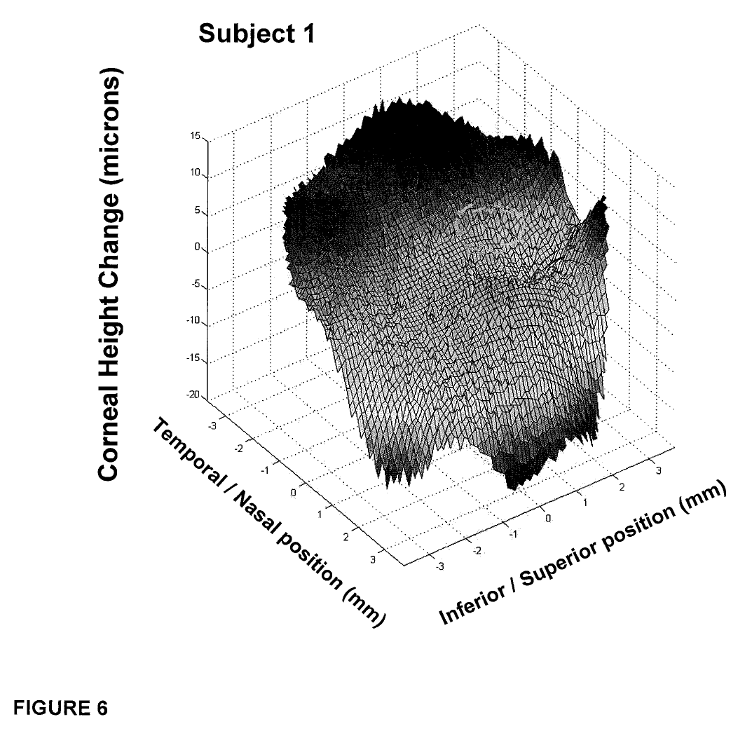

In FIG. 2, both the portion of the anterior corneal surface directly above the inlay 260 and the extended drape region 255 are controlled by the physical shape of the inlay 210. FIGS. 4 through 6 present examples of the anterior corneal height change in human subjects implanted with three different inlay designs. The height change in microns was derived from analysis of post-operative and pre-operative corneal topographic maps, recorded with a Pentacam (Oculus Inc.) topographer. Table 1 summarizes the inlay design parameters and corresponding corneal height changes. Subjects 1 through 3 correspond to FIGS. 4 through 6, respectively.

TABLE 1
Correlation of Anterior Corneal Surface Change with Inlay Design

| | Inlay | Inlay | Inlay | Inlay | | Corneal | Effect |
|---|---|---|---|---|---|---|---|
| | Posterior | Anterior | Edge | Center | Inlay | Height | Zone |
| | Radius | Radius | Thickness | Thickness | Diameter | Change | Diameter |
| Subject | (mm) | (mm) | (microns) | (microns) | (mm) | (microns) | (mm) |
| 1 | 7.5 | 7.15 | 33 | 35 | 1.5 | 20 | 5.0 |
| 2 | 10 | 6.95 | 11 | 29 | 1.5 | 13 | 3.0 |
| 3 | 10 | 9.5 | 11 | 25 | 1.5 | 7 | 3.2 |

The resulting change to the anterior corneal surface does not reflect a simple geometric projection of the inlay's physical design. The “effect” zone diameter is at least twice the inlay diameter. The corneal height change is less than the inlay center thickness, ranging between 30% and 60% thinner.

If the biomechanical effect followed the simple geometric rule of preserving the inlay thickness profile as discussed in the Background of the Invention, then the expected refractive changes are given in Table 2 and compared to approximations of the actual refractive change from the topography data. The latter assumes the post-operative surfaces are spherical.

TABLE 2
Expected and Estimates of Achieved Clinical Refractive Changes

| | Expected Refractive | Estimated Refractive |
|---|---|---|
| | Change Assuming | Change |
| Subject | Constant Profile | from Measured Topographies |
| 1 | 2 D | 2.4 D |
| 2 | 17 D | 4.4 D |
| 3 | 2 D | 2.1 D |

In general the expected spherical refractive change (ΔRx) is given by:

$$Δ Rx = ( {n}_{c} - 1 ) ( \frac{1}{{r}_{anterior}} - \frac{1}{{r}_{posterior}} ) \qquad Equation 1$$

The estimated spherical refractive change from the topographic data is given by:

$$Δ Rx = \frac{8 ( {n}_{c} - 1 )}{{dia}_{Effect}^{2}} ( CT ) \qquad Equation 2$$

Here n_{c} is the index of the cornea (1.376), dia_{Effect} is the diameter of the inlay effect, CT is the center thickness, rₐₙₜₑᵣᵢₒᵣ and rₚₒₛₜₑᵣᵢₒᵣ are the inlay's anterior and posterior radii of curvature.

In Table 2, the actual refractive effect is a factor of one to four less than the expected refractive effect. Furthermore, no apparent correlation between Tables 1 and 2 easily explains this difference in actual versus expected refractive effects. This example highlights that the biomechanical effects are complicated and must be at least partially understood to effectively design intracorneal inlays.

This biomechanical effect also suggests that inlays with an index of refraction substantially different than that of the cornea, may not be advantageous for small diameter intracorneal inlays for the correction of presbyopia. If the inlay's index of refraction exceeds that of the cornea, the inlay itself contributes additional dioptric power in addition to the anterior corneal surface changes patent application Ser. No. 11/381,056, titled “Design Of Inlays With Intrinsic Diopter Power,” filed on May 1, 2006, outlines the technical contribution of each dioptric component. Because the inlay's intrinsic diopter power is a function of the inlay's anterior surface curvature and because that curvature may be more curved than expected from non-biomechanical design methods (as explained above), the inlay with intrinsic power may cause two unwanted issues. First, the intrinsic power may exceed the near power, bringing the near focus point of a near object too close for comfortable function. Second, the increased curvature and intrinsic power may add significant positive spherical aberration, reducing the image quality of the near focus provided by the inlay. Additionally, since the inlay diameter is smaller than the pupil, there is the potential for light scattering from the inlay edge, which occurs only when the index of refraction of the inlay material is substantially different than the index of the surrounding cornea media. Clinically, implanted subjects may report glare or halos when looking at bright light sources at night.

A method for designing a small inlay to provide near vision according to an embodiment will now be given.

(1) The first step is to determine the maximum effect zone (d_{eff}) that is an acceptable tradeoff between the near vision improvement and the loss of distance vision. Considerations include the pupil size of the specific subject or a group of characteristic subjects (e.g., subjects within a particular age range) while reading or viewing nearby objects, and the pupil size for distance viewing, especially at night. Based on the analysis of pupil sizes recorded in subjects with various intracorneal inlays, consideration of the distance and near visual acuities, review of literature on pupil size changes, and supplemental theoretical ray-trace analysis of theoretical eyes with the intracorneal inlay, the preferred embodiment is for an effect zone no less than about 2.0 mm in diameter and no more than about 4.0 mm in diameter—for inlays intended for the correction of presbyopia by means of a multifocal optical effect. In an exemplary application, the inlay is placed in one eye to provide near vision while distance correction by other means is performed on both the inlay eye and the fellow eye. In this example, both eyes contribute to distance vision, with the non-inlay eye providing the sharpest distance vision. The eye with the inlay provides near vision.

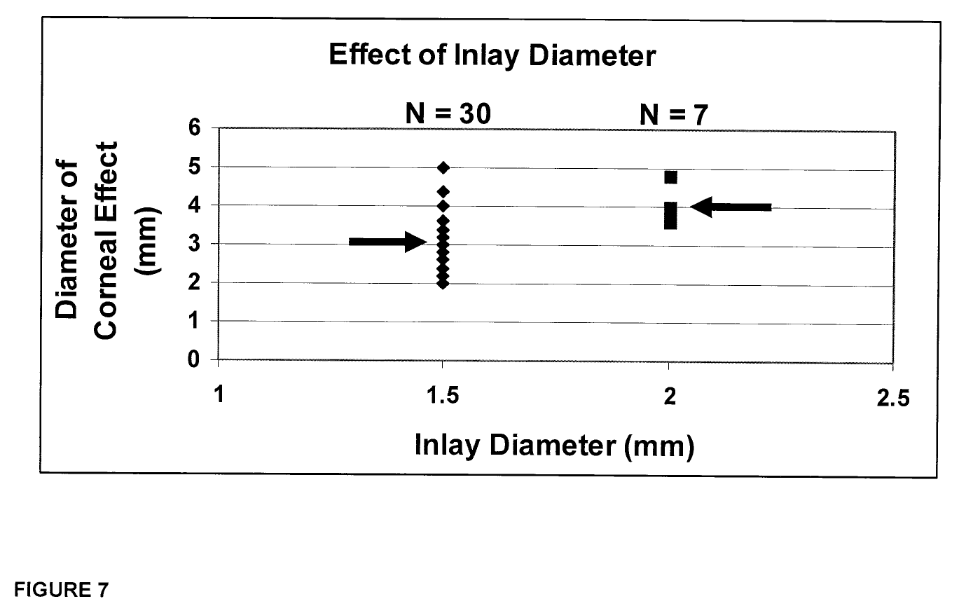

(2) The second step is to determine the inlay diameter. In theory, the “effect” zone increases with inlay diameter (FIG. 2). To determine the optimal inlay diameter, sophisticated Finite Element Analysis (FEA) models, based on the physical and mechanical properties of the cornea, can be studied. Alternatively, empirical experiments measuring the change in actual corneal surfaces as a function of different inlay designs can be conducted, either in vitro or in vivo. FIG. 7 presents the corneal effect diameters for a 1.5 mm and a 2 mm diameter inlay design derived from topographic analysis similar to those shown in FIGS. 4-6. This empirical in vivo biomechanical model demonstrates that the diameter of the “effect” zone is close to twice the inlay's physical diameter. The spread of the data points show that the biomechanics of the inlay and cornea interaction is complicated and depends on a number of variables. In an exemplary design, we target an “effect” zone diameter of about 3.0 mm. Thus, the exemplary inlay diameter is about 1.5 mm.

(3) The third step is to determine the inlay's posterior radius of curvature. In FIG. 2, the intracorneal inlay 210 rests on a lamellar bed 280, which is the anterior aspect of the cornea underneath the flap. The posterior curvature of the inlay 220 must match the curvature of the lamellar bed to avoid gaps which can facilitate biochemical changes such as keratocyte activation leading to optical opacities. For inlays which are “stiffer” (i.e., greater modulus of elasticity) than the cornea, pre-operative estimates of the bed curvature are used to select the inlay with the appropriate posterior curvature. In the preferred embodiment, the inlay is more flexible (i.e., modulus of elasticity less than or equal to about 1.0 MPa) than the cornea (corneal modulus about 1.8 MPa), and the inlay will bend and conform to the bed curvature when placed under the flap. Analysis of the posterior shape of the implanted inlays by means of Optical Coherence Topography suggests that bed radius of curvature ranges between 6 mm and 9 mm. To avoid any possibility of a gap beneath the inlay, the preferred inlay posterior radius of curvature is 10 mm.

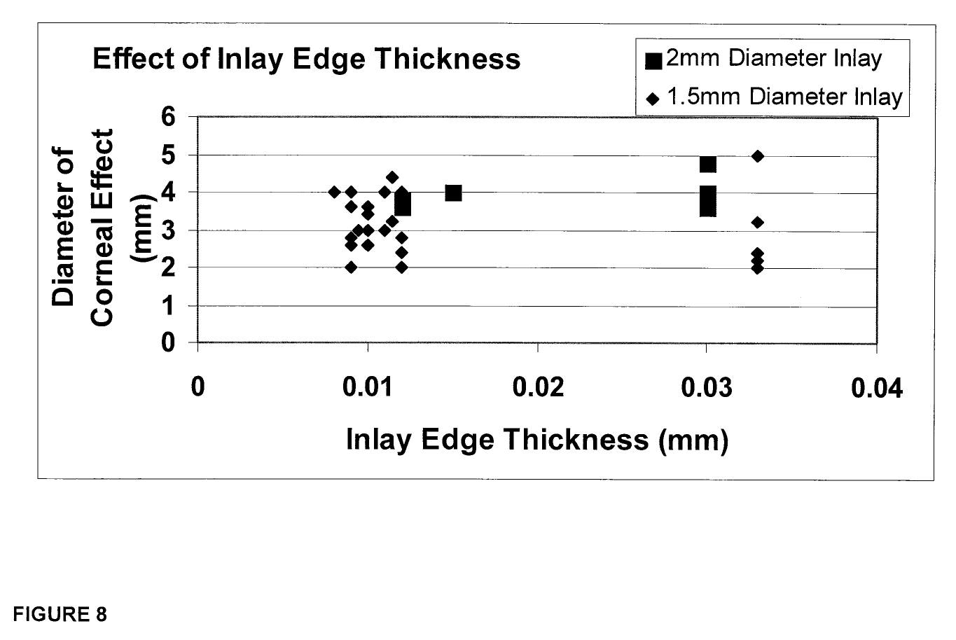

(4) The fourth step is to determine the inlay edge thickness. In FIG. 2, the finite edge thickness 250 creates a gap under the flap at the peripheral edge of the inlay, potentially leading to biochemical changes resulting in opacities in the cornea. Thus, the edge thickness is minimized and preferably is less than about 20 microns. FIG. 8 presents corneal effect diameters as a function of two inlay groups with different edge thicknesses, for the 1.5 mm and 2 mm diameter inlays. This in vivo biomechanical model demonstrates that the edge thickness does not significantly alter the corneal effect diameter. In the preferred embodiment, the edge thickness is 10 microns. The edge thickness 250 can not be effectively zero, because the inlay must be manipulated when packaged and when placed on the cornea using a variety of instruments. The edges of an inlay with effectively zero edge thickness will wrap or roll due the finite electrostatic surface forces. It is particularly difficult to unroll such a delicate inlay without damage.

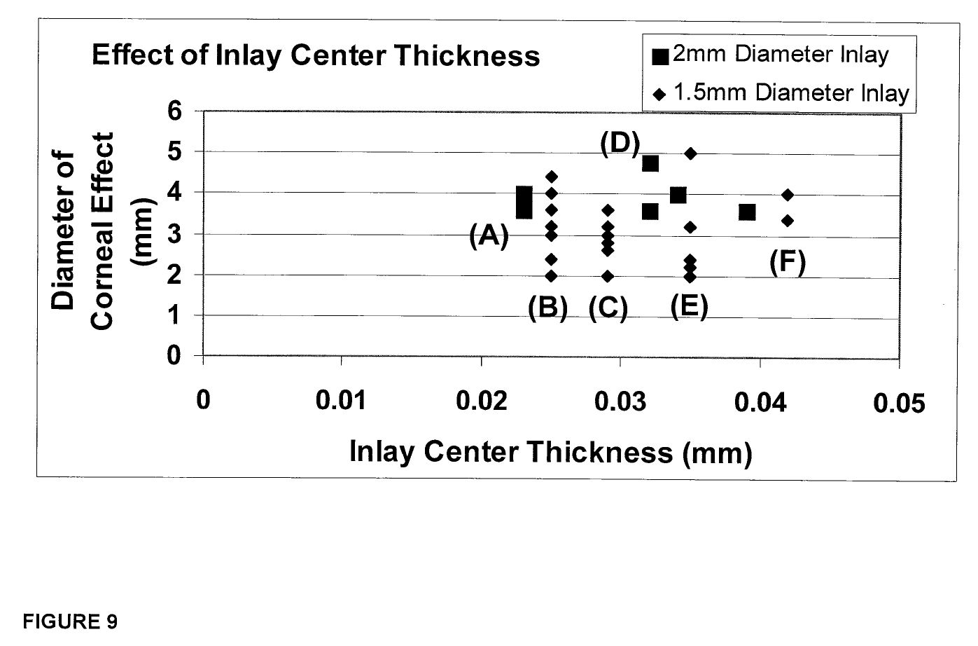

(5) The fifth step is to determine the inlay center thickness. In theory (FIG. 2) and in practice (Table 2), the “effect” zone increases with increased inlay center thickness 265, in at least these selected subjects. However, FIG. 9 demonstrates that this relationship does not exist across all inlays of different diameters, edge thicknesses and center thicknesses. The biomechanical response of the anterior surface is more complicated. FIG. 9 plots the corneal effect diameter as a function of the inlay center thickness, for six different combinations of inlay diameter, center thickness and edge thickness; these are labeled by A-F. The design parameters are listed in Table 3.

TABLE 3
Nominal Inlay Designs with Clinical Outcome Data

| | Poster | Anterior | | | |
|---|---|---|---|---|---|
| | Radius of | Radius of | Inlay | Inlay Edge | Inlay Center |
| | Curvature | Curvature | Diameter | Thickness | Thickness |
| Design | (mm) | (mm) | (mm) | (microns) | (microns) |
| A | 10 | 9.50 | 2.0 | 13 | 21 |
| B | 10 | 9.50 | 1.5 | 11 | 25 |
| C | 10 | 6.95 | 1.5 | 10 | 29 |
| D | 7.5 | 5.40 | 2.0 | 30 | 34 |
| E | 7.5 | 7.15 | 1.5 | 33 | 35 |
| F | 10 | 5.40 | 1.5 | 10 | 42 |

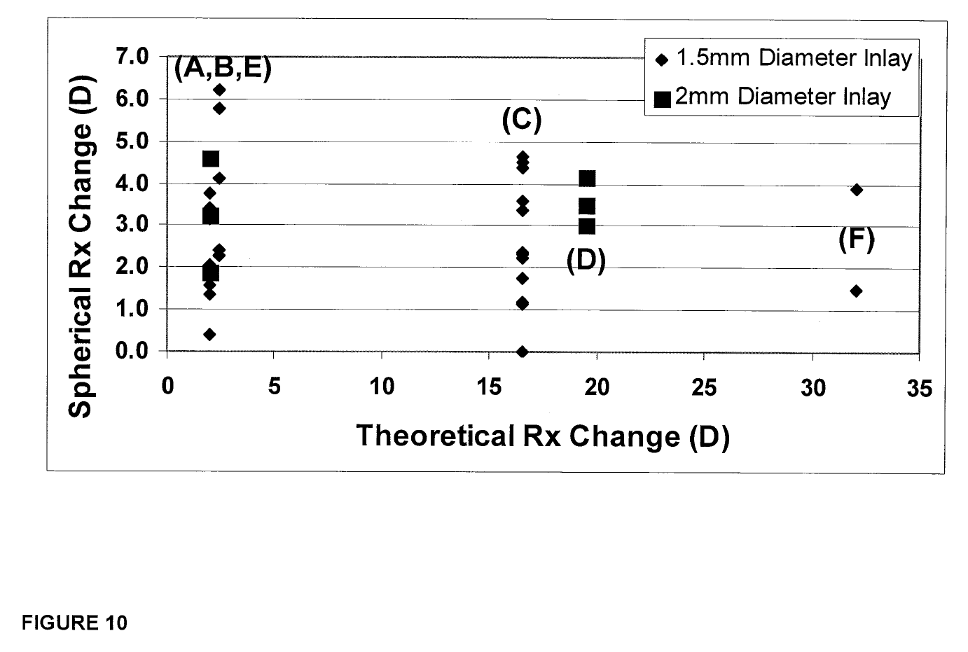

Ultimately, for successful inlay function, the anterior surface of the cornea must steepen the effect-zone shape to give both the desired refractive change and acceptable image quality. FIG. 10 plots estimates of the spherical refractive power change (derived from subject topographies) as a function of the theoretically expected refractive change based on the method of constant thickness profile described in U.S. patent application Ser. No. 11/293644. The theoretical method does not take into account the complex biomechanical effects suggested above. For half of the inlay designs, the theoretical inlay power exceeds the estimates of the achieved refractive change, demonstrating that the biomechanical response requires inlays with designs more curved than the anterior surface (e.g., higher theoretical refractive change) to achieve the desired anterior surface change.

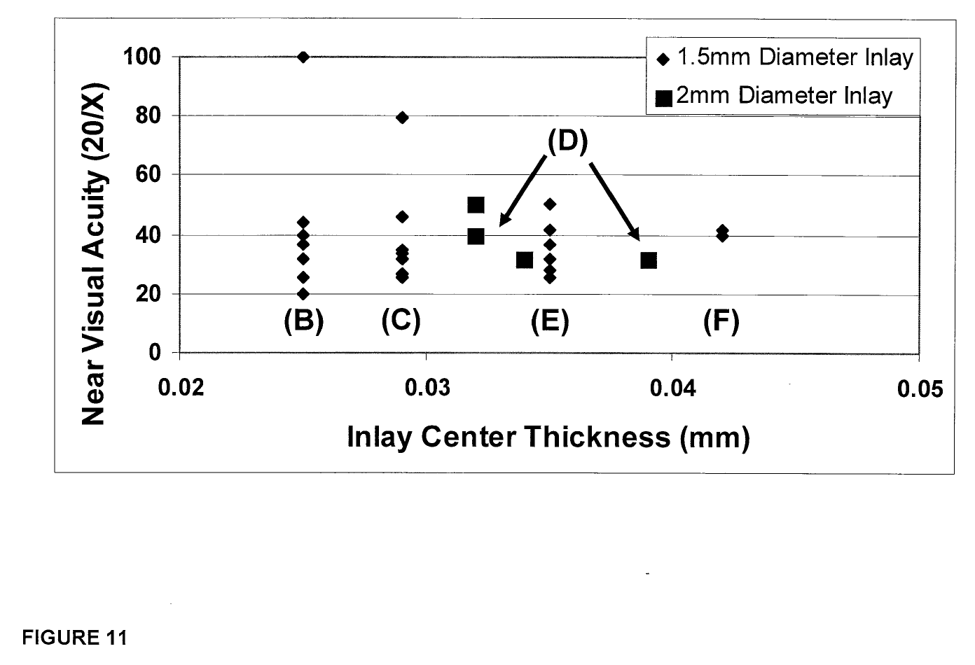

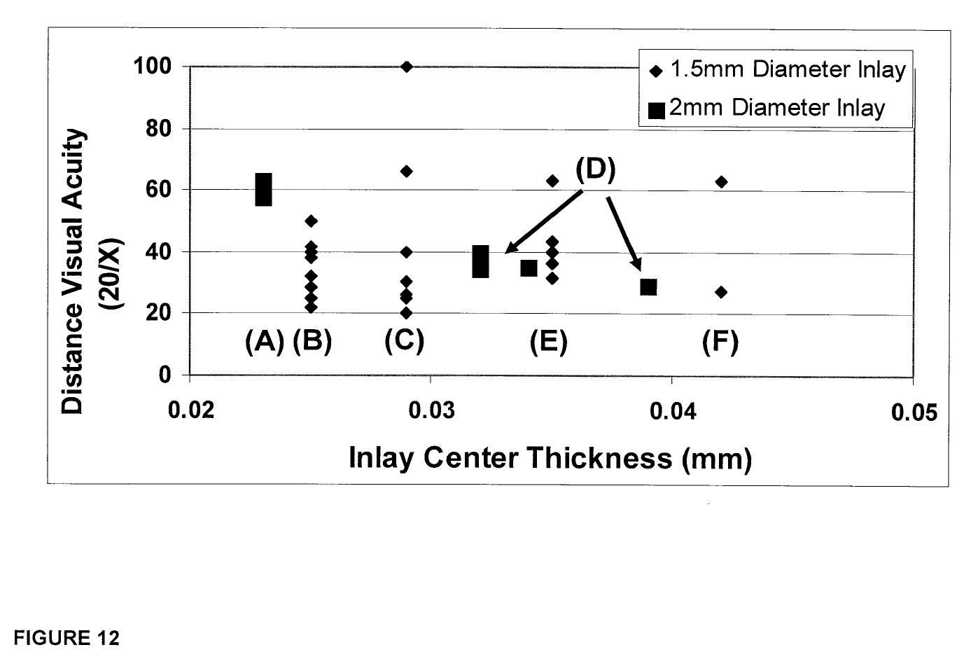

FIGS. 11 and 12 assess the effect of inlay design on image quality. A key clinical measure of image quality is visual acuity (VA), which is the smallest angular height of a standardized “letter” that is just recognized. Visual acuity is measured in units of 20/X, where 20/20 is considered normal vision, 20/40 is marginally acceptable vision, and 20/50 or larger (X) is considered poor vision. The inlay effect is designed to improve near vision. FIG. 11 plots the near visual acuity (without refractive correction) as a function of the inlay center thickness for the different inlay designs. Only designs B through E create anterior surface changes which provide acceptable near image quality (i.e., near VA generally better than 20/40). FIG. 12 plots the distance visual acuity (without refractive correction) as a function of the inlay center thickness for the different inlay designs. Likewise, the anterior surface changes induced by designs B through E provide acceptable distance image quality (i.e., distance VA generally better than 20/40). The failure of design “A” may simply be the lack of sufficient test cases. Thus, inlays with center thickness ranging between about 20 microns and 50 microns alter the anterior corneal surface, providing the desired refractive change and image quality for distance and near vision. The preferred embodiment is design “C” because it provides the best near and distance VA, excluding outlying cases.

It may be desirable to obtain inlay designs with different clinical refractive effect ranges. For early presbyopic subjects (e.g., 40 to 45 years) who retain substantial accommodation, a clinical effect in the range of 0.5 to 1.5 diopters may be more appropriate. For presbyopes with no accommodation (e.g., over about 55 years), a clinical effect in the range of 2 diopter to 3 diopters may be more desirable. To more consistently achieve refractive changes in these ranges, a clinical “nomogram” must be developed, based on a large series of clinical implant cases. A “nomogram” empirically correlates the desired refractive change as a function of the inlay design parameters (e.g., inlay center thickness) and surgical parameters (e.g., flap thickness). The surgeon uses the desired outcome and surgical parameters to select the best inlay design parameters, based on the nomogram.

(6) The sixth step is to determine the inlay anterior radius of curvature. In FIG. 2, the inlay's anterior surface shape and curvature 215 influence the shape and curvature of the portion of the cornea's anterior surface 240 above the inlay 260 and the drape region 255 extending beyond 260. A biomechanical model derived from either theoretical FEA modeling or empirically from in vivo or in vitro experiments must be used to set the inlay's anterior surface shape and curvature 215, given the desired anterior corneal shape 240. The above discussion for the inlay thickness (section (5)), based on the empirical in vivo biomechanical mode demonstrated that designs “B” through “E” produced observed dioptric changes and distance and near image quality in the desired range of performance. Thus, the range of acceptable small diameter inlay anterior radius of curvatures falls between about 5.0 mm and 10.0 mm (see Table 3). However, the preferred embodiment is design “C”, with a spherical inlay anterior radius of curvature of 6.95 mm.

More generally and especially when more sophisticated and analytic models of the inlay and cornea biomechanical response are known, non-spherical anterior inlay surfaces 215 may be desirable. Such aspheric anterior inlay surfaces may be either flatter or steeper compared to a spherical surface.

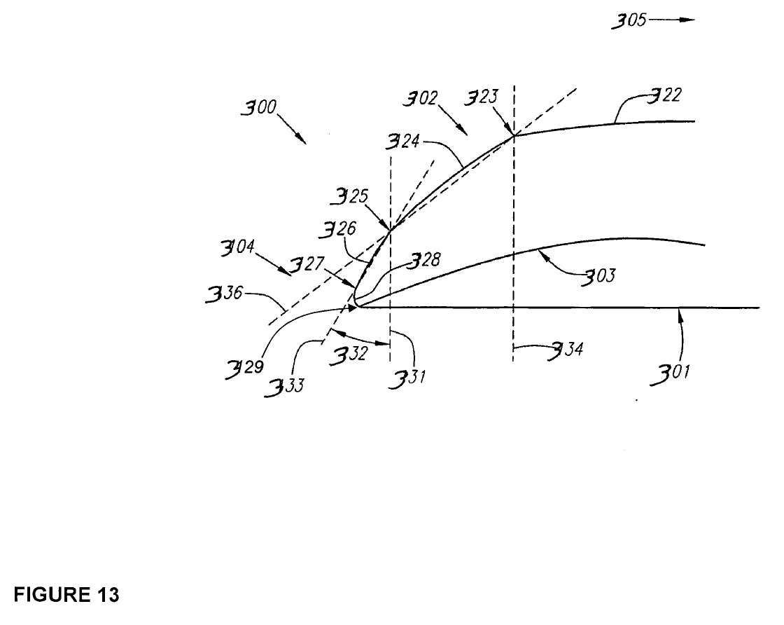

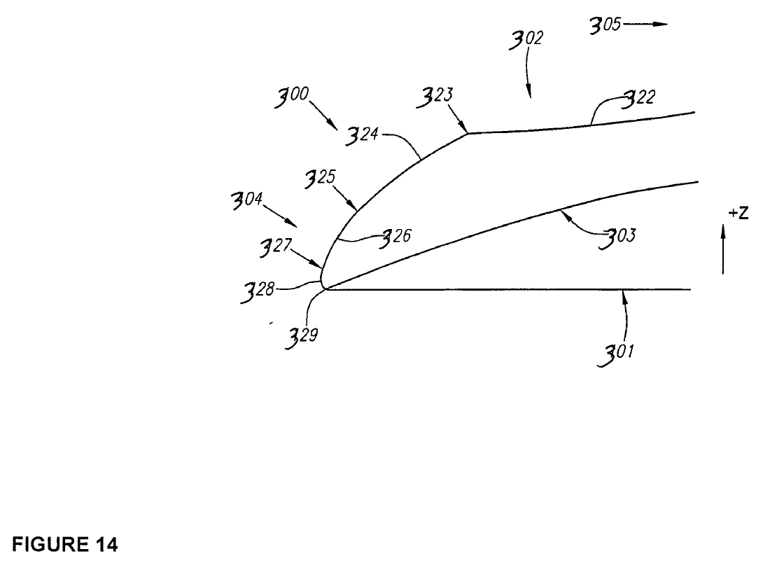

(7) The seventh step is to determine the edge taper. A magnified view of the inlay edge is shown in FIG. 13. The inlay's posterior surface 303 meets the inlay's edge 328 at the periphery 329. The inlay's anterior surface is composed of the peripheral taper 300 (regions 324 and 326), and the central region 322. The central region 322 comprises most of the inlay's anterior surface area. The taper design may taken on a variety of shapes, as shown in FIGS. 14 through 19, and may consist of one (e.g., 324) or two (e.g., 324 and 326) segments. The segments (324 and 326) may be curved or straight. The inlay edge 328 may also be curved or straight.

The shape of the taper region 300 and the curvature of the central region 322 control how fast the anterior surface drape returns to the original anterior corneal surface and influences the anterior corneal surface's drape zone shape. The drape zone shape in turn determines the distribution of dioptric powers in the drape zone region and the retinal image quality for primarily intermediate and near objects. The subtleties of the taper zone shape become most important when a sophisticated biomechanical model of the inlay and corneal interaction has been derived.

Mechanically, a taper region 300 is required to maintain sufficient material volume in the inlay periphery, creating a semi-rigid peripheral ring. The peripheral ring is needed for the inlay to maintain its shape when stored in solution and minimize the tendency of the inlay edge to curl, which significantly interferes with inlay manipulation in manufacturing and during surgical placement. The taper region 300 is also required when it is necessary to increase the inlay's center thickness while maintaining a constant inlay diameter, edge thickness, and posterior radius of curvature. Increasing the center thickness causes the central anterior surface 322 to curve more and may become too curved, base on the biomechanical model employed. To allow for a flatter central anterior surfaces 322, a taper region 300 (324 and 326) connects the taper-central junction point 323 to the peripheral edge 328 at the taper-edge junction 327. The taper has the additional advantage of creating “sidedness”. The taper on only the anterior surface refracts light differently when the inlay is anterior face up compared to posterior face up. Thus, the surgeon can visually determine if the inlay is in the proper orientation when placed in the cornea.

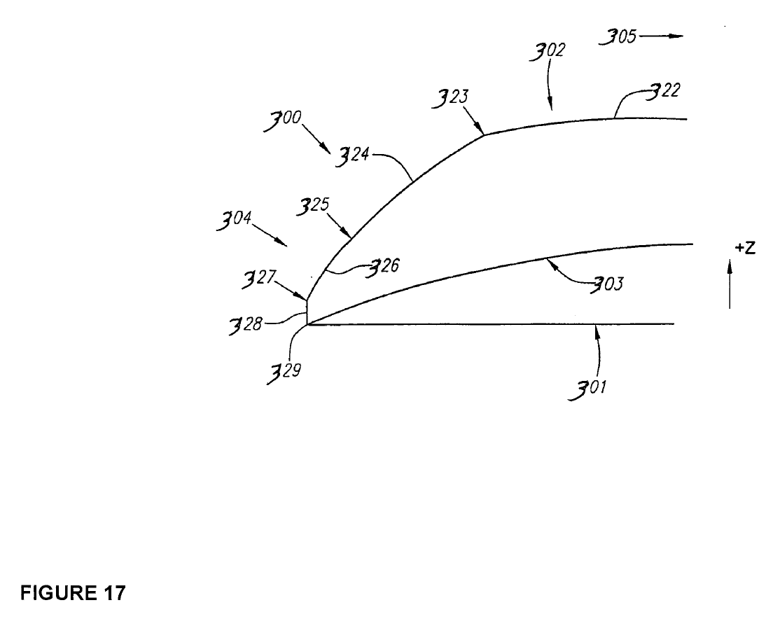

In the preferred embodiment (FIG. 17), an exemplary taper design for the 1.5 mm diameter inlay design “C” in Table 3 consists of the following dimensions: a) a taper-central junction 323 at a radius 0.699 mm with respect to the central Z axis, b) a first taper region 324, called the “bevel”, with a radius of curvature 3.000 mm, and c) a second taper region 326 specified by the edge slope angle 332 (FIG. 13) of 25 degrees and a second taper region 326 radius of curvature 0.05 mm.

More generally, the edge and taper design may take on different forms, depending primarily on the edge thickness and curvature of the central region 322. The first taper region's 324 radius of curvature may vary in the range of about 1-10 mm. The second taper region's 326 radius of curvature may vary in the range of about 0.001 to 1 mm, with an edge slope angle 332 between 0 and 90 degrees. These ranges are for illustrative purposes only and in no way limit the embodiments described herein.

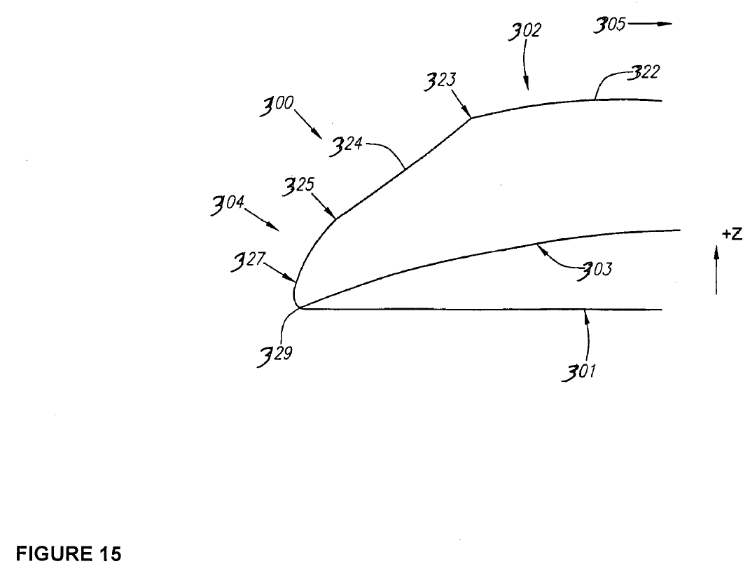

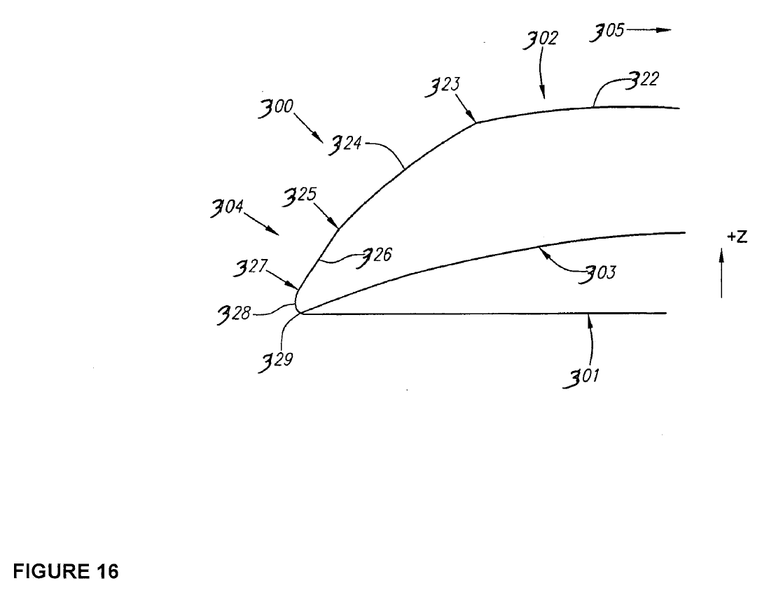

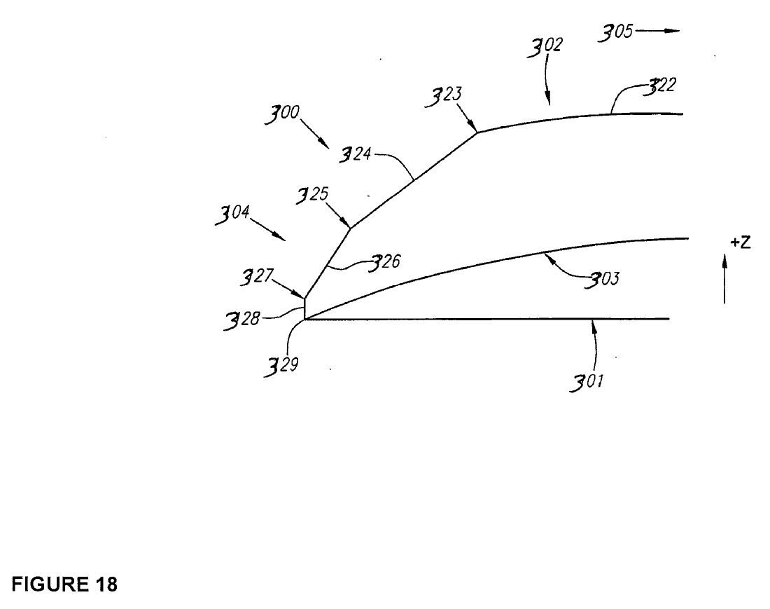

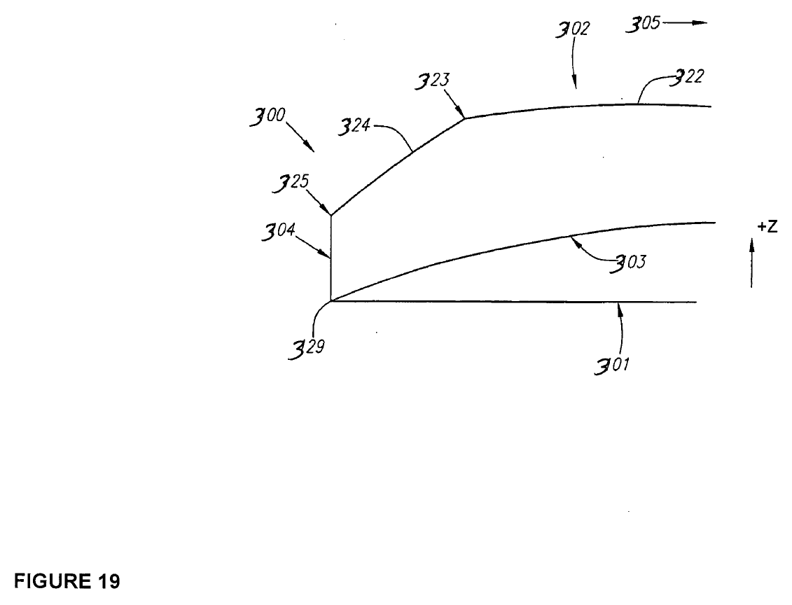

In the embodiment depicted in FIG. 14, anterior surface central region 322 is substantially aspherical. The rate of curvature of aspherical surfaces typically decreases or increases as the surface progresses outwards towards taper-central junction 323. In this embodiment, the curvature of aspheric surface 322 is flatter near the taper-central junction 323 compared to the anterior surface curvature at the center of the inlay. In the embodiment depicted in FIG. 15, beveled portion 324 is flat. In the embodiment depicted in FIG. 16, second taper region 326 is flat. Any combination of flat and curved surfaces can be implemented. For example, in FIG. 18, beveled portion 324, the second taper region 326, and the edge 328 are all flat. Also, the taper region 300 may consist of only one region. For example in FIG. 19, only a flat bevel exists, without a second taper region., and the edge 304 is flat. More details of these edge taper designs can be found, e.g., in U.S. patent application Ser. No. 11/106,983, filed on Apr. 15, 2005, the specification of which is herein incorporated by reference.

(8) The final step is to iterate for the optimal design. Where accurate, predictive, and preferably analytic biomechanical models exist, connecting the change in anterior corneal surface shape to the inlay's physical design, more sophisticated optical outcomes can be targeted. For example, the drape zone 255 (FIG. 2) transitions in power between the near power and distance power, providing some power for intermediate distances. Thus, if the biomechanical model accurately predicts small changes in the drape zone, one can use optical analysis such as optical ray-trace programs to derive the optimal drape zone surface shape to maximize intermediate distance vision while retaining acceptable near and distance vision in the central and peripheral zones, respectively. One then uses the biomechanical model and Steps (2) through (7) to derive the ideal inlay design. In practice, the ideal inlay design may not be achievable, because of biomechanical restriction. Thus, one iterates steps (1) through (7) to achieve the best inlay design most closely approximating the ideal optical design.

The above steps apply to other inlay designs not included in the clinical tests reported above. Those tests (e.g., FIGS. 11 and 12) demonstrate that a range of inlay designs (e.g., Table 3) provide acceptable distance and near vision (e.g., visual acuity). Therefore, the precise shape of the inlay's surfaces may be less of importance compare to the volume of this very small object placed beneath the much larger and thicker flap. The inlay may serve as a “post”, causing a “tenting” of the overlying flap which increases the curvature of the anterior corneal surface, increasing the refractive power in the central region. Thus, inlays with volumes similar to those inlay designs in Table 3, but with flat anterior and posterior surfaces should also effectively provide acceptable distance and near visual quality. The inlay's anterior and posterior radii of curvature may function to optimize the balance of distance and near visual quality, while providing some intermediate visual quality by control of the shape of the drape 255 (FIG. 2) region.

### Large Diameter Intracorneal Inlays for the Correction of Refractive Error

For the correction of distance refractive error (e.g., hyperopia), it is desirable that the “effect” zone be typically larger than the maximum pupil size typically experienced by the intended implant subject. This condition minimizes the possibility of out-of-focus peripheral light rays from distance objects which are out-of-focus on the retina. Such conditions may lead to unwanted glare phenomena from oncoming headlights while driving at night. Typically, low light (e.g., twilight) pupil sizes range from about 4 mm to about 8 mm for middle aged and older subjects.

A second consideration is the size of the keratometric flap needed to allow placement of the inlay in the cornea. Typical flap diameters range from about 8 mm to 10 mm, and place a physical limit on the maximum size of the “effect” zone.

A method for designing a large diameter inlay to provide refractive error according to an embodiment will now be given.

(1) The first step is to determine the minimum “effect” zone (d_{eff}) that is an acceptable tradeoff between the need to reduce the potential for nighttime glare and the finite size of the keratometric flap. In the preferred embodiment, the nominal flap diameter of 9 mm fixes the “effect” zone at a diameter of 9 mm. This is significantly larger than low light pupil size and thus minimizes the potential for nighttime glare.

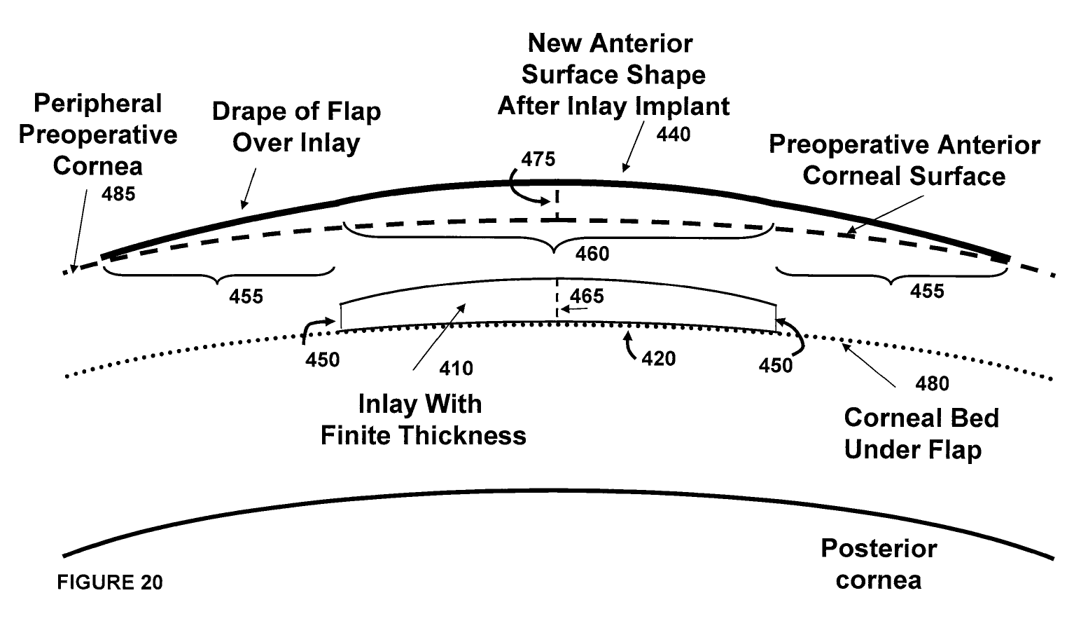

(2) The second step is to determine the inlay diameter. The methods discussed in Small Diameter Intracorneal Inlays for the Correction of Presbyopia apply to larger diameter inlays. Given a desired “effect” zone of 9 mm in diameter, the empirical biomechanical model based on a small diameter inlay suggests a minimum inlay diameter of about 4.5 mm. In FIG. 20, the surface curvature in the drape zone 455 transitions from the necessary curvature for the desired refractive correction in the central region 460 to the lower dioptric power in the peripheral cornea 485. Thus, only the central aspect of the drape region 455 contributes to correction of the desired refractive error. To maximize the size of the optical zone providing the desired refractive change, in the preferred embodiment, the diameter of the inlay is increased to 5 mm, which extends the total optical zone to something more than 5 mm, further reducing the potential for nighttime glare effects. The inlay diameter can not be increased much more, because the finite thickness of the anterior corneal surface profile 440 will interfere with the peripheral margin of the flap diameter. The interference may provide an additional force “lifting” the flap at the flap margin, causing a gap and subsequent intrusion of epithelial cells into the corneal stroma. This effect can lead to serious post-operative complications.

(3) The third step is to determine the inlay's posterior radius of curvature, The same discussion outlined in Small Diameter Intracorneal Inlays for the Correction of Presbyopia applies to larger diameter inlays. In the preferred embodiment, the large diameter “flexible” inlay's posterior curvature 420 conforms to the corneal bed 480. Given that the corneal bed radius of curvature can range from 6 mm to 9 mm, an exemplary inlay posterior radius of curvature of 10 mm is needed to prevent the possibility of gaps beneath the inlay when placed on the corneal bed.

(4) The fourth step is to determine the inlay's edge thickness. The same discussion outlined in Small Diameter Intracorneal Inlays for the Correction of Presbyopia applies to larger diameter inlays. However, because the large diameter inlay has significantly more mass, a slightly larger edge thickness with an exemplary inlay thickness of 15 microns is needed to enhance the mechanical stability of the peripheral edge.

(5) The fifth step is to determine the inlay's edge taper. The same discussion outlined in Small Diameter Intracorneal Inlays for the Correction of Presbyopia applies to larger diameter inlays. The preferred inlay edge taper design is similar to the small diameter intracorneal inlay, with the modification that the large diameter inlay is slightly larger, with an exemplary thickness of 15 microns.

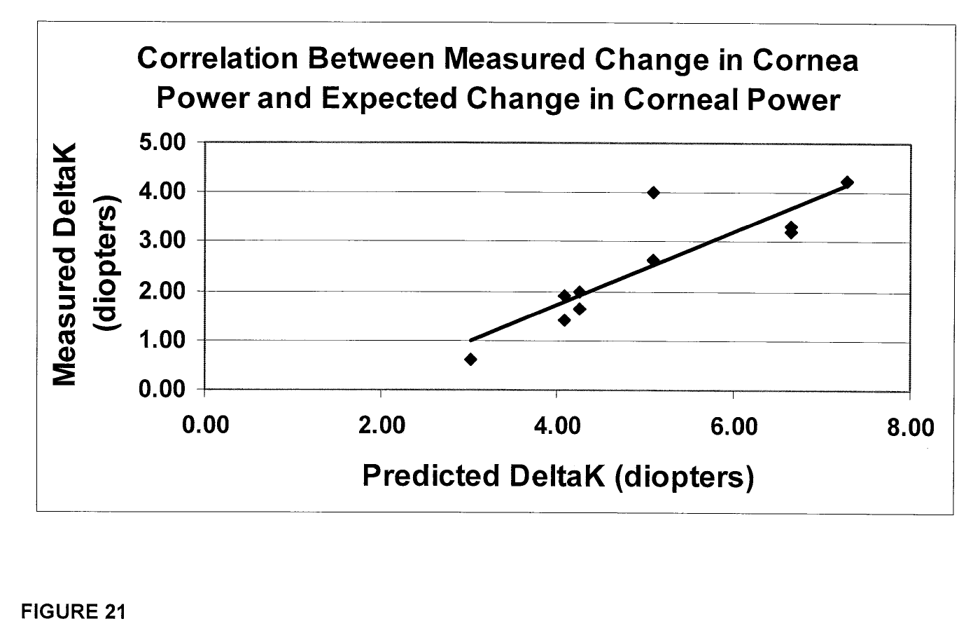

(6) The sixth step is to determine the inlay center thickness and inlay anterior radius of curvature. In FIG. 20, the volume of the large diameter (e.g. 5 mm, providing 3 diopters of correction) inlay 410 is approximately 20 times larger than the volume of a 1.5 mm diameter inlay. The 5 mm diameter inlay volume is about 36% of the volume of the flap volume in the inlay “effect” zone. The 1.5 mm diameter inlay volume is about 4% of the flap volume in the inlay “effect” zone. For these examples, the flap geometry assumes a 43D cornea, 8.5 mm bed radius, a 120 micron flap thickness, and an 8 mm diameter effect zone for the 5 mm inlay. Thus, the physical shape of the larger diameter inlay 410 has more of a role in determining the shape of the resulting post-operative anterior corneal surface 440, compared to the effect with a smaller diameter inlay. The biomechanical relationship between the measured change in the corneal power (based on the change in manifest optometric refraction) and the theoretically expected change in corneal power is show in FIG. 21. The data is from 10 subjects implanted with a 5 mm diameter inlay for the correction of hyperopic error. The theoretically expected change in corneal power is derived from the inlay's physical design by the method outlined in U.S. patent application Ser. No. 11/293,644, titled “Design of Intracorneal Inlays, ” filed on Dec. 1, 2005, the entirety of which is incorporated herein by reference. Key elements of the design process are discussed below in Initial Design of Large Diameter Intracorneal Inlays. FIG. 21 demonstrates that the measured change in corneal power is less than the expected change in corneal power, and the two are linearly related. Thus, for the large diameter inlay, to incorporate the biomechanical response of the cornea, one begins with the desired change in corneal power (e.g., “measured”), determines the expected (e.g. theoretical) change in corneal power based on FIG. 21, and calculates the inlay shape by the methods of initial inlay design discussed below. The conversion between the expected (theoretical) and desired (measured) change in corneal power is derived from the linear fit in FIG. 21: theoretical (D)=1.3×desired+1.75.

Given the exemplary physical parameters determined for the large diameter inlay in Steps (1) through (5), this method for the design of the large diameter inlay is implemented to generate the anterior radius of curvature and center thicknesses (Table 4) in the exemplary application for the correction of various amounts of hyperopic refractive error. Note that the optical zone without the edge taper is assumed to be 4.8 mm in diameter.

TABLE 4
Design of Large Diameter Intracorneal Inlays

| | | Anterior Radius | Inlay Center |
|---|---|---|---|
| | | Curvature | Thickness |
| | Rx | (mm) | (microns) |
| Inlay Parameters: | 1 | 9.24 | 39 |
| Diameter (mm) = 4.80 | 1.5 | 9.09 | 44 |
| Edge Thickness (microns) = 0.015 | 2 | 8.93 | 49 |
| Posterior Rad Curv (mm) = 10 | 2.5 | 8.79 | 55 |
| | 3 | 8.64 | 60 |
| | 3.5 | 8.50 | 66 |
| | 4 | 8.36 | 72 |
| | 4.5 | 8.22 | 77 |
| | 5 | 8.09 | 83 |
| | 5.5 | 7.95 | 89 |
| | 6 | 7.82 | 95 |

The center thicknesses of the low diopter correction, large diameter inlays, fall in the same range as the center thickness of the small diameter inlays for the correction of presbyopia. However, at higher diopters, the large diameter inlays are significantly thicker. Thus, the biomechanical response of the anterior cornea for the large diameter inlays will be different compared to biomechanical response of the small diameter inlays.

(7) The last step is to iterate for the optimal design. Where accurate, predictive, and preferably analytic biomechanical models exist, connecting the change in anterior corneal surface shape to the inlay's physical design, more sophisticated optical outcomes can be targeted. For example, it may be desired that the peripheral portion of the central inlay affect 460 (FIG. 20) and the shape of the drape region 455 be designed to have a slightly flatter aspheric profile to correct or reduce higher order spherical aberration of the total eye. If the biomechanical accurately predicts small changes in these regions, one can use optical analysis such as optical ray-trace programs to derive the optimal surface shape to minimize the total eye spherical aberration. One then uses the biomechanical model and Steps (1) through (6) to derive the ideal inlay design. In practice, the ideal inlay design may not be achievable, because of biomechanical restriction. Thus, one iterates steps (1) through (6) to achieve the best inlay design most closely approximating the ideal optical design.

### Initial Design of Large Diameter Intracorneal Inlays

The fundamental assumption for the initial large diameter inlay design is that the thickness profile of the desired change on the anterior corneal surface is equal to the inlay's physical thickness profile. The first step is to determine the cornea's anterior radius of curvature, r′ₐ, that provides the desired refractive change (ΔRx ). The equivalent change in the cornea's refractive power, ΔK_{equiv}, at the anterior surface is given by:

$$Δ {K}_{equiv} = \frac{1}{\frac{1}{Rxdist} - V} \qquad Equation 2$$

where V is a spectacle vertex distance, e.g., 0.012 meters, from the spectacle plane to the cornea's anterior surface. The spectacle vertex distance, V, takes into account that measurements of the cornea's refractive power are typically taken with a spectacle located a distance from the cornea's anterior surface, and translates these power measurements to the equivalent power at cornea's anterior surface.

The pre-implant refractive power at the anterior corneal surface may be approximated by Kavg−Kpost, where Kavg is the average corneal refractive power within approximately the optical zone created by the inlay and Kpost is a posterior corneal refractive power. The desired radius of curvature, r′ₐ, of the anterior surface may be given by:

$${r}_{a}^{′} = \frac{( 1.376 - 1 )}{( Kavg - Kpost + Δ {K}_{equiv} )} \qquad Equation 3$$

For purposes of design, Kpost may be approximated as −6 diopters. The pre-implant radius of curvature, rₚᵣₑᵢₘₚₗₐₙₜ, may be approximated by:

rₚᵣₑᵢₘₚₗₐₙₜ=(1.376−1)/(Kavg−Kpost) Equation 4

The two radius of curvatures need not originate from the same origin.

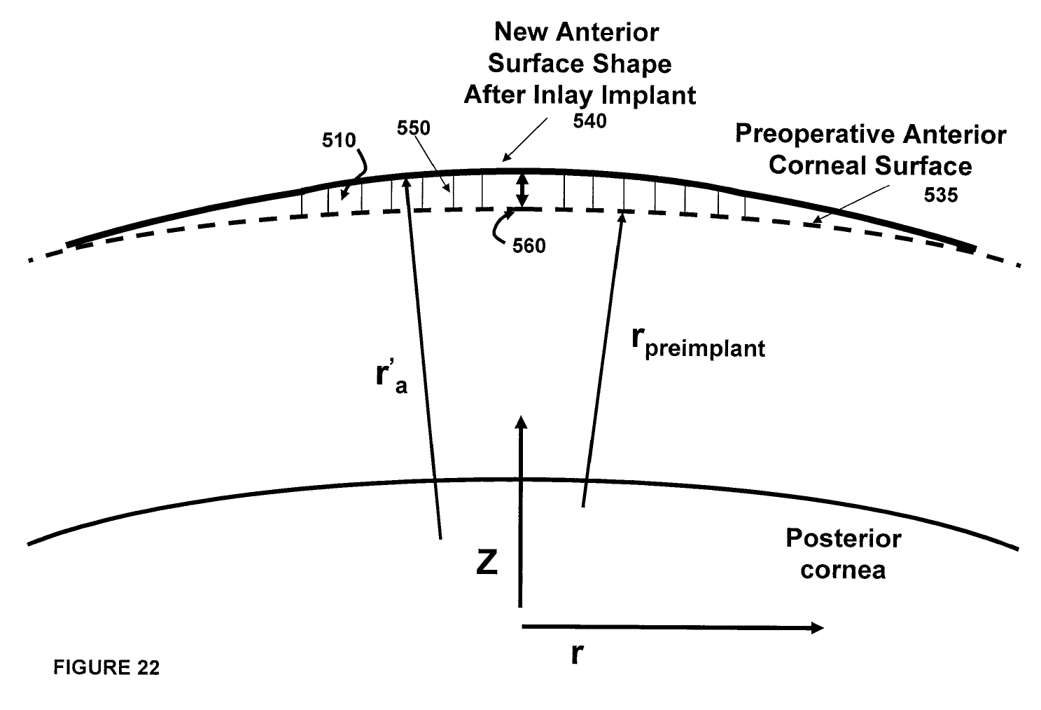

FIG. 22 shows a cross-sectional view of a thickness profile 510 specified by a difference between the desired anterior corneal surface 540 and the pre-implant anterior corneal surface 535. In FIG. 22, lines 550 pointing from the pre-implant anterior surface 535 to the desired anterior surface 540 represent the axial thickness, L(r), of the thickness profile 510 at different positions along an r axis that is substantially perpendicular to an optical z axis. The double arrow 560 represents a center thickness, L_{c}, of the thickness profile. In this embodiment, the thickness profile 510 is rotationally symmetric about the z axis. Note that for the initial design, the thickness profile extends only to the projection (460 in FIG. 20) directly above the inlay (410 in FIG. 20).

The thickness L(r) of the thickness profile may be given by:

L(r)=L_{c}+Zₚᵣₑᵢₘₚₗₐₙₜ(r;rₚᵣₑᵢₘₚₗₐₙₜ)−Z_{anew}(r;r′ₐ) and

L_{c}=Z_{anew}(d₁/2)−Zₚᵣₑᵢₘₚₗₐₙₜ(d₁/2) Equation 5

where L_{c} is the center thickness of the thickness profile, Zᵢₘₚₗₐₙₜ(r) is the pre-operative anterior corneal surface as a function of r, Z_{anew} (r) is the desired anterior corneal surface as a function of r, and d₁ is the diameter of the inlay. In the example above, the anterior surfaces Z_{anew} and Zₚᵣₑᵢₘₚₗₐₙₜ were assumed to be spherical. This need not be the case. The anterior surfaces may also be aspheric. More generally, the desired anterior surface Z_{anew} may be a function of desired refractive change (ΔRx) and also more complex design parameters, e.g., an aspheric surface for higher-order aberration correction. Also, the pre-implant anterior surface Zₚᵣₑᵢₘₚₗₐₙₜ is generally aspheric. For designs requiring aspheric surfaces, the surface function Z(r) may be given by the general aspheric form:

$$Z ( r ) = \frac{\frac{{r}^{2}}{{r}_{c}}}{1 + \sqrt{1 - ( 1 + k ) {( \frac{r}{{r}_{c}} )}^{2}}} + {a}_{4} {r}^{4} + {a}_{6} {r}^{6} \qquad Equation 6$$

where: r_{c} is the radius of curvature

k is a conic constant

a₄ and a₆ are higher order aspheric constants

For a spherical surface, k=0, a₄=0, and a₆=0. The human cornea may be approximated by k=−0.16, a₄=0 and a₆=0. The radius of curvature, r_{c} is specified as describe above (e.g., rₐ or rₚᵣₑᵢₘₚₗₐₙₜ), and the other parameters may specify corrections for higher-order aberrations.

The above expressions for the thickness profile are intended to be exemplary only. Other mathematical expressions or parameters may be used to describe similar or other thickness profiles. Therefore, the invention is not limited to particular mathematical expressions or parameters for describing the thickness profile.

After the required thickness profile L(r) is determined, the inlay is dimensioned to have substantially the same thickness profile. The profiles should have the same thickness to within about one micron, which would cause a diopter difference of about one eight of a diopter if the center thickness differs by one micron. An eight of a diopter is half the accuracy with which ophthalmic refractive errors are manually recorded. Next, the thickness profile of the inlay is increased by the finite edge thickness 450 (FIG. 20) by the manufacturing process. This finite edge thickness is one factor inducing the drape 455 as illustrated in FIG. 20. This comprises the initial large-diameter inlay design for the correction of refractive error. This initial design method is incorporated in Steps 6 and 7 of “Large Diameter Intracorneal Inlays for the Correction of Refractive Error,” wherein the biomechanical model discussed is used to determine the final design.

The initial design method above assumed that the index of refractive of the inlay is the same as the cornea, in which case changes in refractive power of the cornea is due solely to the change in the anterior corneal surface induced by the inlay. An inlay with intrinsic power (e.g., a higher index of refraction than the cornea) may also be used, in which changes in the refractive power is provided by a combination of the physical inlay shape and the intrinsic power (i.e., index of refraction) of the inlay. Design methods for inlays with intrinsic power is described in application Ser. No. 11/381,056, titled “Design Of Inlays With Intrinsic Diopter Power, ” filed on May 1, 2006, the entirety of which is incorporated herein by reference.

In the foregoing specification, the invention has been described with reference to specific embodiments thereof. It will, however, be evident that various modifications and changes may be made thereto without departing from the broader spirit and scope of the invention. As another example, each feature of one embodiment can be mixed and matched with other features shown in other embodiments. As yet another example, the order of steps of method embodiments may be changed. Features and processes known to those of ordinary skill may similarly be incorporated as desired. Additionally and obviously, features may be added or subtracted as desired. Accordingly, the invention is not to be restricted except in light of the attached claims and their equivalents.

### Appendix A

### Manufacture of Inlay Material

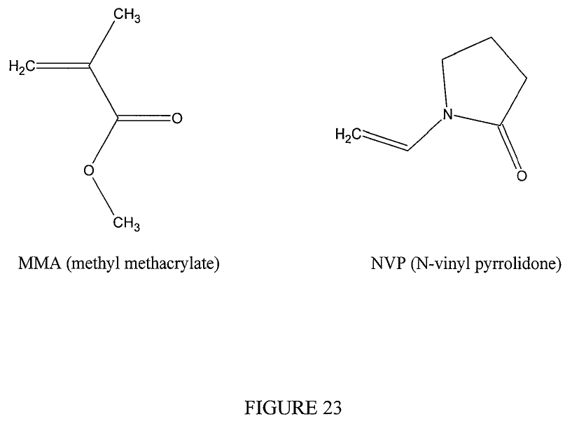

In a preferred embodiment, the inlay material is a random block large copolymer consisting of 78% NVP (normal-vinyl pyrrolidone) and 22% MMA (methyl methacrylate). The polymerization is a free radical reaction with AIBN (azobisisobutyronitrile) used as an initiator. The amount of initiator is about 0.5%. Allymethacrylate is used as the crosslinker. The amount of crosslinker is about 0.23%. The chemical structures of the monomer precursors for the inlay material are shown in FIG. 23.

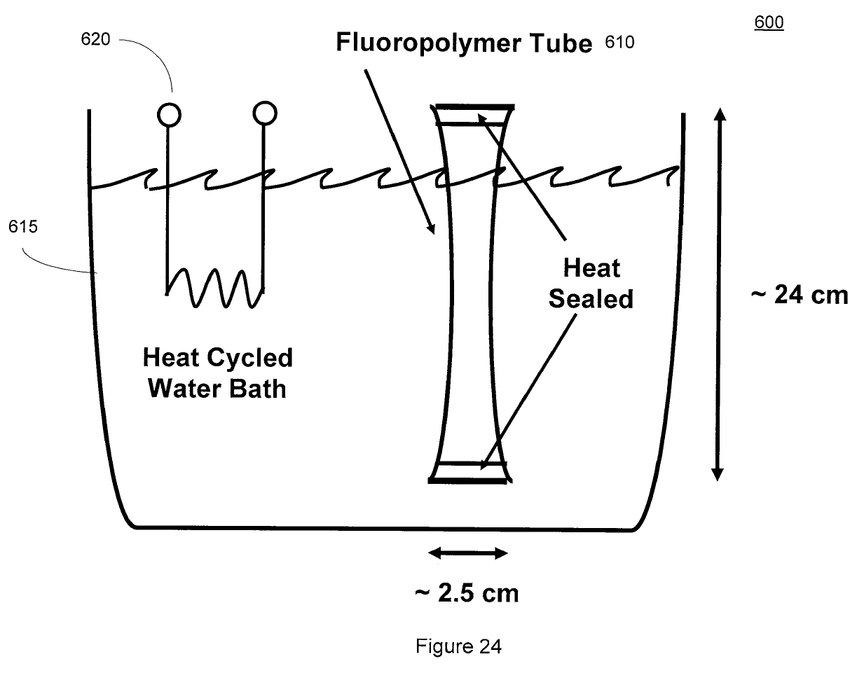

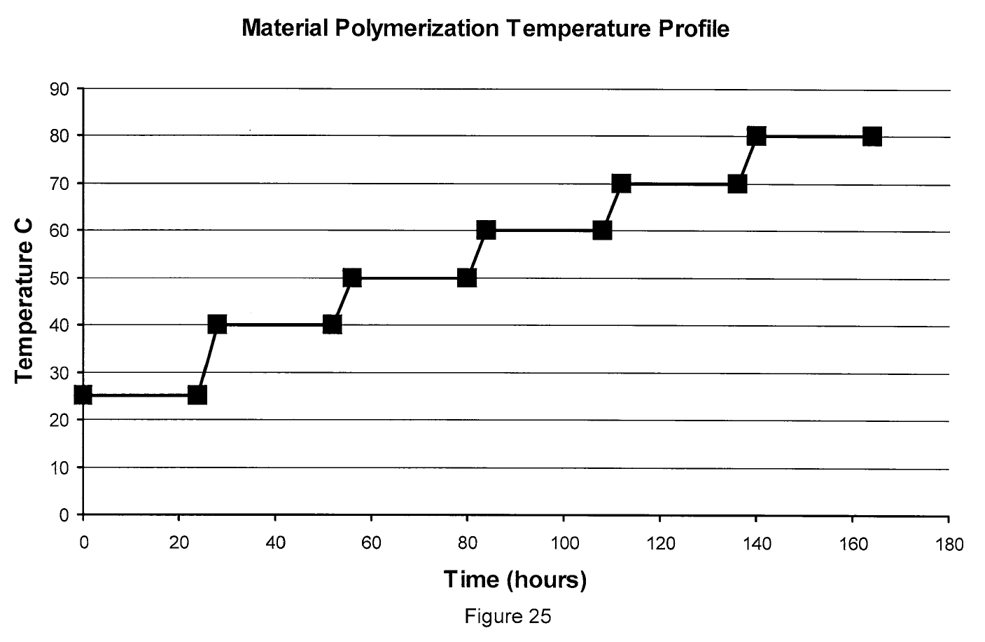

The monomer constituents are mixed together along with initiator and crosslinker in a batch process. The mixture is then poured into a fluoropolymer tube. The mixture can be the composition described above or other previously described material. FEP (fluorinated ethylene propylene) is the preferred material although tube can be made out of other fluoropolymers. Fluoropolymer tube materials are advantageous because they do not contain fatty acids (palmitate, oleate, and stearate), DBS (dodecylbenzene sulfonate), PDMS (poly(dimethyl siloxane)) and other contaminates resulting in the manufacture of polypropylene. These contaminates are absorbed by the hydrogel inlay material which may illicit a biological response after implantation. The sealed FEP tube containing the monomer mixture is placed into a heated water bath. The polymerization is exothermic and water bath is used to control the rate of reaction. In the preferred embodiment, the polymerization is a 6 day process where the temperature in the water bath is increased in approximately 10° C. increments from 25° C. to 80° C. FIG. 24 shows an exemplary apparatus 600 for carrying out the polymerization. The apparatus 600 includes a water bath 615 and a heater 620 for controllably heating the water bather 615. FIG. 24 also shows the a sealed fluoropolymer tube 610 containing the monomer mixture placed in the water bath 615. FIG. 25 is a temperature chart showing the temperature of the water bath over time for the polymerization process. The time and temperatures selected in FIG. 25 were optimized based on porosity, reaction kinetics and manufacturing cycle. The temperature ramp described in FIG. 25 is the preferred embodiment, however the temperature steps can vary from 5-15° C. The ramp time between temperatures can vary from 1 to 24 hours. The hold time at a specific temperature can range from 1 to 48 hours. The overall polymerization temperature/temperature ramp can vary from 110° C. to 95° C.

After polymerization the material rod is removed from the FEP tube. The polymer in the shape of a rod is either lathe cut or centerless ground to ensure roundness. The rod is then cut into buttons approximately ½ long. In both operations (grinding and cutting) mineral oil is used as a lubricant to cut the polymer material. The button is placed in the lathe collet and base curve is cut. The button is removed from the collet and blocked on an arbor using a water soluable wax. The button/arbor assembly is placed in the lathe machine and the front curve is cut.

The finished lens is decanted into water filled vial. The lens is inspected dimensionally. The lens is autoclaved followed by a multi step extraction process. The extraction process removes any residual water soluble NVP monomer and initiator. After extraction the inlay is packaged and steam sterilized.

In the preferred embodiment, the finished product is a hydrogel with a water content of approximately 80%. It is transparent with a UV light transmittance of about 95%. The refractive index is 1.3718±0.008, which is substantially the same as the refractive index of the cornea.

1. A method for treating presbyopia using an intracorneal inlay, comprising:
altering the shape of the anterior surface of a cornea to correct near vision by implanting the inlay in the cornea, wherein the inlay has anterior and posterior surfaces, an edge surface between the anterior and posterior surfaces, and a diameter of approximately between 0.75 mm and 3 mm;
wherein distance vision is provided by a region of the cornea located peripheral to an area of the anterior surface of the cornea altered by the inlay.

2. The method of claim 1, wherein the inlay has a diameter of approximately between 0.75 mm and 2 mm.

3. The method of claim 1, wherein the inlay has an index of refraction substantially equal to the index of refraction of the cornea.

4. The method of claim 1, wherein the inlay has a center thickness of between 10 and 100 microns.

5. The method of claim 1, wherein the inlay induces a change in the anterior surface of the cornea having a diameter of between 1.5 mm to 4 mm in diameter.

6. The method of claim 1, wherein the inlay induces a change in the anterior surface of the cornea having a diameter that is at least twice the diameter of the inlay.

7. The method of claim 1, wherein the anterior surface of the inlay has a radius of curvature of between 5 mm and 10 mm.

8. The method of claim 1, wherein the anterior surface of the inlay has a radius of curvature greater than 5 mm.

9. The method of claim 1, wherein the anterior surface of the inlay has a radius of curvature of approximately 6.95 mm.

10. The method of claim 1, wherein the posterior surface of the inlay conforms to the shape of a corneal surface on which the inlay is placed.

11. The method of claim 10, wherein the inlay has a modulus of elasticity equal to or less than the modulus of elasticity of the cornea.

12. The method of claim 10, wherein the inlay has a modulus of elasticity equal to or less than 1.0 MPa.

13. The method of claim 1, wherein the inlay has a modulus of elasticity greater than the modulus of elasticity of the cornea.

14. The method of claim 13, wherein the posterior surface of the inlay substantially matches the shape of a corneal surface on which the inlay is placed.

15. The method of claim 1, wherein the edge surface has a thickness of less than 20 microns.

16. The method of claim 1, wherein the edge surface has a thickness of less than 15 microns.

17. The method of claim 1, wherein the edge surface has a thickness of approximately 10 microns.

18. The method of claim 1, wherein the posterior surface of the inlay has a radius of curvature of at least 6 mm.

19. The method of claim 1, wherein the posterior surface of the inlay has a radius of curvature of about 10 mm.

20. The method of claim 1, wherein the anterior surface of the inlay has a central region and a tapered region, the tapered region being located between the central region and the edge surface.

21. The method of claim 20, wherein the tapered region has a first portion interfacing to the central region and a second portion interfacing to the edge surface.

22. The method of claim 21, wherein the first portion has a radius of curvature of between 1 mm and 10 mm, and the second portion has a radius of curvature of between 0.001 mm to 1 mm.

23. The method of claim 1, wherein the inlay induces a change in the anterior surface of the cornea having a radius of curvature that is greater than the radius of curvature of the anterior surface of the inlay.

24. The method of claim 1, wherein the inlay is implanted at a depth of 80 to 180 microns beneath the anterior surface of the cornea.

25. The method of claim 1, wherein the inlay is implanted at a depth of 100 to 140 microns beneath the anterior surface of the cornea.

26. The method of claim 1, wherein the inlay induces a change in the anterior surface of the cornea having a diameter greater than the diameter of the inlay.

27. The method of claim 1, wherein the inlay induces a change in height of the anterior surface of the cornea that is less than the center thickness of the inlay.

28. The method of claim 1, wherein the inlay reduces a radius of curvature of the anterior surface of the cornea.

29. The method of claim 1, wherein the area of the anterior surface of the cornea altered by the cornea includes a central area and an intermediate area between the central area and the peripheral region of the cornea, and wherein the central area provides near vision and the intermediate area provides intermediate vision.

30. The method of claim 1, further comprising:
prior to implantation of the inlay, performing a corrective procedure on the cornea to correct distance vision.

31. The method of claim 30, wherein the corrective procedure to correct distance vision comprises a LASIK procedure.

32. The method of claim 31, further comprising:
cutting a flap in the cornea during the LASIK procedure; and
after the LASIK procedure, reopening the flap and implanting the inlay beneath the flap.

33. The method of claim 1, further comprising performing a corrective procedure to correct distance vision concurrently with implantation of the inlay.

34. The method of claim 33, wherein the corrective procedure to correct distance vision comprises a LASIK procedure.

35. The method of claim 34, further comprising:
cutting a flap in the cornea during the LASIK procedure; and
implanting the inlay beneath the flap.

36. The method of claim 1, further comprising:
cutting a flap in the cornea;
lifting the flap to expose an interior of the cornea;
placing the inlay in the interior of the cornea; and
repositioning the flap over the inlay.

37. The method of claim 1, further comprising:
cutting a pocket in the stroma of the cornea; and
placing the inlay in the pocket.

38. The method of claim 1, wherein the inlay comprises NVP (N-vinyl pyrrolidone) and MMA (methyl methacrylate).

39. The method of claim 38, wherein the inlay comprises approximately 78% NVP (N-vinyl pyrrolidone) and approximately 22% MMA (methyl methacrylate).

40. The method of claim 1, wherein the inlay comprises 20% to 50% HEMA (hydroxyethyl methylacrylate), 85% to 30% NVP (normal vinyl pyrrolidone), and/or 0% to 25% PVP (polyvinyll pyrrolidone).

41. The method of claim 1, wherein the inlay comprises 15% to 50% MMA (methyl methylacrylate), 85% to 30% NVP (normal vinyl pyrrolidone), and/or 0% to 25% PVP (polyvinyll pyrrolidone).

42. The method of claim 1, further comprising:
determining an initial design of the inlay; and
iterating the initial design to obtain an optimal design.

43. A method for correcting visional impairment, comprising:
implanting an inlay in a cornea, the inlay having anterior and posterior surfaces, an edge surface between the anterior and posterior surfaces, and a diameter, wherein the inlay induces a change in the anterior surface of the cornea having a diameter greater than the diameter of the inlay.

44. The method of claim 43, wherein the diameter of the change in the anterior surface of the cornea is at least 1.5 times greater than the diameter of the inlay.

45. The method of claim 43, wherein the diameter of the change in the anterior surface of the cornea is at least twice the diameter of the inlay.

46. The method of claim 43, wherein the inlay has an index of refraction substantially equal to the index of refraction of the cornea.

47. The method of claim 43, wherein the diameter of the inlay is between 4 mm and 6 mm.

48. The method of claim 43, wherein the inlay has a center thickness of between 20 and 130 microns.

49. The method of claim 43, wherein the anterior surface of the inlay has a radius of curvature between 6.5 mm and 10 mm.

50. The method of claim 43, further comprising:
multiplying a desired refractive change value by a coefficient;
adding a refractive change offset to the desired refractive change value multiplied by the coefficient to obtain a modified refractive change value; and
selecting a thickness for the inlay based on the modified refractive change value.

51. The method of claim 50, where the coefficient is about 1.3.

52. The method of claim 50, where the refractive change offset is about 1.75 diopters.

53. The method of claim 43, wherein the posterior surface of the inlay conforms to the shape of a corneal surface on which the inlay is placed.

54. The method of claim 53, wherein the inlay has a modulus of elasticity equal to or less than the modulus of elasticity of the cornea.

55. The method of claim 53, wherein the inlay has a modulus of elasticity equal to or less than 1.0 MPa.

56. The method of claim 43, wherein the inlay has a modulus of elasticity greater than the modulus of elasticity of the cornea.

57. The method of claim 43, wherein the posterior surface of the inlay substantially matches the shape of a corneal surface on which the inlay is placed.

58. The method of claim 43, wherein the edge surface has a thickness of less than 20 microns.

59. The method of claim 43, wherein the edge surface has a thickness of less than 15 microns.

60. The method of claim 43, wherein the edge surface has a thickness of approximately 10 microns.

61. The method of claim 43, wherein the posterior surface of the inlay has a radius of curvature of at least 6 mm.

62. The method of claim 43, wherein the posterior surface of the inlay has a radius of curvature of about 10 mm.

63. The method of claim 43, wherein the anterior surface of the inlay has a central region and a tapered region, the tapered region being located between the central region and the edge surface.

64. The method of claim 63, wherein the tapered region has a first portion interfacing to the central region and a second portion interfacing to the edge surface.

65. The method of claim 64, wherein the first portion has a radius of curvature of between 1 mm and 10 mm, and the second portion has a radius of curvature of between 0.001 mm to 1 mm.

66. The method of claim 43, wherein the inlay induces a change in the anterior surface of the cornea having a radius of curvature that is greater than the radius of curvature of the anterior surface of the inlay.

67. The method of claim 43, wherein the inlay is implanted at a depth of 80 to 180 microns beneath the anterior surface of the cornea.

68. The method of claim 43, wherein the inlay is implanted at a depth of 100 to 140 microns beneath the anterior surface of the cornea.

69. The method of claim 43, wherein the inlay comprises NVP (N-vinyl pyrrolidone) and MMA (methyl methacrylate).

70. The method of claim 69, wherein the inlay comprises approximately 78% NVP (N-vinyl pyrrolidone) and approximately 22% MMA (methyl methacrylate).

71. The method of claim 43, wherein the inlay comprises 20% to 50% HEMA (hydroxyethyl methylacrylate), 85% to 30% NVP (normal vinyl pyrrolidone), and/or 0% to 25% PVP (polyvinyll pyrrolidone).

72. The method of claim 43, wherein the inlay comprises 15% to 50% MMA (methyl methylacrylate), 85% to 30% NVP (normal vinyl pyrrolidone), and/or 0% to 25% PVP (polyvinyll pyrrolidone).

73. The method of claim 43, further comprising:
determining an initial design of the inlay; and
iterating the initial design to obtain an optimal design.

74. The method of claim 43, further comprising:
cutting a flap in the cornea;
lifting the flap to expose an interior of the cornea;
placing the inlay in the interior of the cornea; and
repositioning the flap over the inlay.

75. An intracorneal inlay, comprising:
a biocompatible hydrogel material having anterior and posterior surfaces, an edge surface between the anterior and posterior surfaces, and a diameter of approximately between 0.75 mm and 3 mm, wherein the material has an index of refraction substantially equal to the index of refraction of a cornea.

76. The inlay of claim 75, wherein the diameter of the inlay is approximately between 0.75 mm and 2 mm.

77. The inlay of claim 76, wherein the anterior surface of the inlay has a radius of curvature of between 5 mm and 10 mm.

78. The inlay of claim 75, wherein the anterior surface of the inlay has a radius of curvature greater than 5 mm.

79. The inlay of claim 75, wherein the anterior surface of the inlay has a radius of curvature of approximately 6.95 mm.

80. The inlay of claim 75, wherein the inlay has a modulus of elasticity equal to or less than the modulus of elasticity of the cornea.

81. The inlay of claim 75, wherein the inlay has a modulus of elasticity equal to or less than 1.0 MPa.

82. The inlay of claim 75, wherein the inlay has a modulus of elasticity greater than the modulus of elasticity of the cornea.

83. The inlay of claim 75, wherein the edge surface has a thickness of less than 20 microns.

84. The inlay of claim 75, wherein the edge surface has a thickness of less than 15 microns.

85. The inlay of claim 75, wherein the edge surface has a thickness of approximately 10 microns.

86. The inlay of claim 75, wherein the posterior surface of the inlay has a radius of curvature of at least 6 mm.

87. The inlay of claim 75, wherein the posterior surface of the inlay has a radius of curvature of about 10 mm.

88. The inlay of claim 75, wherein the anterior surface of the inlay has a central region and a tapered region, the tapered region being located between the central region and the edge surface.

89. The inlay of claim 88, wherein the tapered region has a first portion interfacing to the central region and a second portion interfacing to the edge surface.

90. The inlay of claim 89, wherein the first portion has a radius of curvature of between 1 mm and 10 mm, and the second portion has a radius of curvature of between 0.001 mm to 1 mm.

91. The inlay of claim 75, wherein the material comprises NVP (N-vinyl pyrrolidone) and MMA (methyl methacrylate).

92. The inlay of claim 91, wherein the material comprises approximately 78% NVP (N-vinyl pyrrolidone) and approximately 22% MMA (methyl methacrylate).

93. The inlay of claim 75, wherein the material comprises approximately 20% to 50% HEMA (hydroxyethyl methylacrylate), 85% to 30% NVP (normal vinyl pyrrolidone), and/or 0% to 25% PVP (polyvinyll pyrrolidone).

94. The inlay of claim 75, wherein the material comprises approximately 15% to 50% MMA (methyl methylacrylate), 85% to 30% NVP (normal vinyl pyrrolidone), and/or 0% to 25% PVP (polyvinyll pyrrolidone).

95. The inlay of claim 75, wherein the inlay has a meniscus shape.

96. An intracorneal inlay, comprising:
a biocompatible hydrogel material having anterior and posterior surfaces, an edge surface between the anterior and posterior surfaces, center and edge thicknesses, and a diameter of approximately between 4 mm and 6 mm, wherein the material has an index of refraction substantially equal to the index of refraction of a cornea, and the center thickness is greater than the edge thickness.

97. The inlay of claim 96, wherein the anterior surface of the inlay has a radius of curvature of between 6.5 mm and 10 mm.

98. The inlay of claim 96, wherein the inlay has a center thickness of between 20 and 130 microns.

99. The inlay of claim 96, wherein the inlay has a modulus of elasticity equal to or less than the modulus of elasticity of the cornea.

100. The inlay of claim 96, wherein the inlay has a modulus of elasticity equal to or less than 1.0 MPa.

101. The inlay of claim 96, wherein the inlay has a modulus of elasticity greater than the modulus of elasticity of the cornea.

102. The inlay of claim 96, wherein the edge surface has a thickness of less than 20 microns.

103. The inlay of claim 96, wherein the edge surface has a thickness of less than 15 microns.

104. The inlay of claim 96, wherein the edge surface has a thickness of approximately 10 microns.

105. The inlay of claim 96, wherein the posterior surface of the inlay has a radius of curvature of at least 6 mm.

106. The inlay of claim 96, wherein the posterior surface of the inlay has a radius of curvature of about 10 mm.

107. The inlay of claim 96, wherein the anterior surface of the inlay has a central region and a tapered region, the tapered region being located between the central region and the edge surface.

108. The inlay of claim 107, wherein the tapered region has a first portion interfacing to the central region and a second portion interfacing to the edge surface.

109. The inlay of claim 108, wherein the first portion has a radius of curvature of between 1 mm and 10 mm, and the second portion has a radius of curvature of between 0.001 mm to 1 mm.

110. The inlay of claim 96, wherein the material comprises NVP (N-vinyl pyrrolidone) and MMA (methyl methacrylate).

111. The inlay of claim 110, wherein the material comprises approximately 78% NVP (N-vinyl pyrrolidone) and approximately 22% MMA (methyl methacrylate).

112. The inlay of claim 96, wherein the material comprises approximately 20% to 50% HEMA (hydroxyethyl methylacrylate), 85% to 30% NVP (normal vinyl pyrrolidone), and/or 0% to 25% PVP (polyvinyll pyrrolidone).

113. The inlay of claim 96, wherein the material comprises approximately 15% to 50% MMA (methyl methylacrylate), 85% to 30% NVP (normal vinyl pyrrolidone), and/or 0% to 25% PVP (polyvinyll pyrrolidone).

114. The inlay of claim 96, wherein the inlay has a meniscus shape.
